(19) United States
(12) Patent Application Publication
(10) Pub. No.: US 2025/0345049 A1
(43) Pub. Date: Nov. 13, 2025

(54) MEDICAL DEVICE DELIVERY SYSTEMS, AND ASSOCIATED DEVICES AND METHODS

(71) Applicants: Boston Scientific Scimed, Inc., Maple Grove, MN (US); Boston Scientific Medical Device Limited, Galway (IE)

(72) Inventors: Deepak Kumar Sharma, Muzaffarnagar (IN); Sharath Kumar G., Kanakapura (IN); Subodh Morey, Ponda (IN); Barry Weitzner, Acton, MA (US); Scott Edward Corbeil, Litchfield, NH (US); James J. Scutti, Norwell, MA (US)

(73) Assignees: Boston Scientific Scimed, Inc., Maple Grove, MN (US); Boston Scientific Medical Device Limited, Galway (IE)

(21) Appl. No.: 19/205,616

(22) Filed: May 12, 2025

Related U.S. Application Data
(60) Provisional application No. 63/646,161, filed on May 13, 2024.

(51) Int. Cl. A61B 17/00

(52) U.S. Cl. CPC A61B 17/00234; A61B 2017/00557

(57) ABSTRACT

A system for delivering a delivery device, mounted on an elongate member such as a medical scope, to a target site within a patient's body. The delivery device is separable from the elongate member to be movable fully independently of the elongate member, and may be selectively anchored with respect to tissue. The delivery device may include a scaffold system with one or more expandable elements which engage the delivery device with the exterior of the elongate member or with tissue within the patient's body. The delivery device may be configured to deliver an auxiliary medical device. A medical device may be delivered by the elongate member and may interact with the auxiliary medical device delivered by the delivery device.

### CROSS-REFERENCE TO RELATED APPLICATIONS

This application claims the benefit of priority under 35 U.S.C. § 119 to U.S. Provisional Application No. 63/646, 161, filed May 13, 2024, the entire disclosure of which is hereby incorporated by reference herein for all purposes.

### FIELD

The present disclosure relates generally to the field of medical devices deliverable and usable within a patient's body. In particular, the present disclosure relates to medical devices and systems deliverable into a patient's body via minimally invasive techniques, and associated methods. Even more particularly, the present disclosure relates to systems for delivering an auxiliary medical device into a patient's body with another medical device, and associated devices and methods. The systems and associated devices are movable independently of the other medical device at the delivery site within the patient's body.

### BACKGROUND

Minimally-invasive procedures, such as transluminal, transcatheter, percutaneous, endoscopic, etc., procedures, have various benefits over open surgery. Whereas open surgery requires cutting open the patient's body to gain access into the patient's body and internal anatomical structures, minimally-invasive procedures access a target site within the patient via a natural orifice (or, in some instances, a small incision not considered to constitute an open-surgery cut). Such methods induce little to no bleeding (minimized to no surgical cutting), require minimal if any implements to hold open the access opening, reduce various risks typical of open surgery, reduce recovery times, and have various other benefits over open surgery. Despite the various benefits over open surgery, the limited working space within a patient available for performing minimally-invasive procedures presents various challenges. Often more than one medical instrument, tool, device, etc., is used during a given medical procedure.

However, the lumen of a delivery device for such medical instruments, tools, devices, etc., often does not accommodate more than one medical instrument, tool, device, etc., to be extended therethrough. Therefore, multiple device exchanges may be required during the procedure. Moreover, the maneuverability of the instruments at the target site for the procedure may be limited by the delivery device through which the instrument extends. For instance, during Endo Luminal Surgery (ESL) procedures, a physician may hold and manipulate a medical scope with one hand and manipulate the shaft of the scope with the other hand to get the scope into the appropriate target position. Once the scope is in the desired location, another medical professional (e.g., a technician) may hand over the relevant accessories (snare, radiofrequency knife, forceps, etc.) to the physician who introduces the accessory into the scope. The accessory is positioned such as by torquing the shaft of the accessory and/or the scope handle. The other medical professional investigates (e.g., prepares) the accessory before introducing it into the scope and may also attend to injecting saline or another medium into the accessory based on the physician's instruction and/or the protocol for the procedure being performed. Technicians are generally responsible for actuating the accessory, injecting saline, etc., based on the physician's instruction. Presently, getting the accessory in the right position to perform the procedure is a combination of scope handle manipulation, shaft manipulation, accessory manipulation, accessory torquing, etc. In some aspects, the accessory needs to be actuated to complete the procedure/task. In dissection or resection procedures, the resecting tool goes through the scope, and the traction device (to remove the cut tissue from the imaging device of the scope and/or from the path of the resecting tool) is extended outside, over the scope. Since the traction device is attached to the scope, it is difficult to maintain continuous traction and/or the desired direction of traction during scope movement. As may be appreciated, the limited spaces in which minimally-invasive procedures typically are performed also impacts the maneuverability of various accessories within the body. Moreover, as noted above, the delivery devices, such as medical scopes, have delivery lumens which are limited in size, and often do not accommodate more than one instrument to be moved therethrough. Accordingly, there remains a need for systems and devices, and associated methods, which increase maneuverability of medical devices used to perform a minimally invasive procedure within a patient.

### SUMMARY

This Summary is provided to introduce, in simplified form, a selection of concepts described in further detail below in the Detailed Description. This Summary is not intended to necessarily identify key features or essential features of the claimed subject matter, nor is it intended as an aid in determining the scope of the claimed subject matter. One of skill in the art will understand that each of the various aspects and features of the present disclosure may advantageously be used separately in some instances, or in combination with other aspects and features of the disclosure in other instances, whether or not described in this Summary. No limitation as to the scope of the claimed subject matter is intended by either the inclusion or non-inclusion of elements, components, or the like in this Summary.

In accordance with various principles of the present disclosure, a delivery system, for delivering a medical device within a patient's body, includes a scaffold system configured to be mounted with respect to an elongate member for delivery therewith to a target site within the patient's body, and separable from the elongate member for axial and rotational movement independent of the elongate member; and at least one actuator element operably associated with the scaffold system to shift at least one element of the scaffold system between a delivery configuration in which the scaffold system is engageable with the elongate member to move with the elongate member, and a deployed configuration in which the scaffold system and the elongate member are capable of moving axially and rotationally independently.

In some aspects, the scaffold system includes at least one expandable element configured to shift between a delivery configuration engageable with the elongate member to move with the elongate member, and a deployed configuration in which the expandable element and the elongate member are capable of moving axially and rotationally independently of each other. In some aspects, the at least one expandable element comprises an inner expandable element and an outer expandable element, wherein the inner expandable element and the outer expandable element are independently actuatable between a delivery configuration and a deployed configuration. In some aspects, the scaffold system further comprises a support structure defining a lumen, the inner expandable element positioned along an interior of the support structure and the outer expandable element positioned within the lumen of the support structure and defining a lumen configured to receive an elongate member therethrough.

In some aspects, the delivery configuration of the at least one expandable element is a radially inwardly expanded configuration reducing an inner diameter of the lumen of the at least one expandable element to be engageable with the exterior of the elongate member extended therethrough to move with the elongate member, and the deployed configuration of the at least one expandable element is substantially elongated to increase the inner diameter of the lumen of the at least one expandable element to separate from the elongate member extended therethrough when the at least one expandable element is in the delivery configuration. In some aspects, the at least one expandable element is inflatable into the delivery configuration. In some aspects, the at least one expandable element is bowed radially inwardly in the delivery configuration and substantially elongated in the deployed configuration. In some aspects, the delivery system further includes a support structure configured to be mounted on the elongate member to move therewith, the support structure defining a seat for the at least one expandable element, the at least one expandable element configured to engage and be seated in the seat of the support structure to move with the support structure and the elongate member when the at least one expandable element is in the delivery configuration, and is expanded away from the seat of the support structure to disengage the seat of the support structure to be axially and radially movable with respect to the support structure and the elongate member.

In some aspects, the delivery system further includes an elongate member having a distal end coupled to the scaffold system and a proximal end extending proximal for access by a medical professional. In some aspects, the elongate member is tubular and configured to deliver an auxiliary medical device with the scaffold system, wherein the tubular elongate member is movable with the scaffold system to move the auxiliary medical device axially or rotationally with respect to an elongate member with which the delivery system is delivered.

In accordance with various principles of the present disclosure, a delivery system, configured to deliver an auxiliary medical device within a patient's body, includes a delivery device configured to be delivered with an elongate member configured to deliver a medical device within a patient's body, and to deliver an auxiliary medical device which may interact with the medical device delivered by the elongate member. In some aspects, the delivery device is movable between a delivery configuration in which the delivery device is mounted with respect to an elongate member to move therewith, and a deployed configuration in which the delivery device and the elongate member are axially and rotatably movable with respect to each other.

In some aspects, the delivery device comprises a scaffold system configured to be mounted with respect to an elongate member for delivery therewith to a target site within the patient's body, and separable from the elongate member for movement independent of the elongate member.

In some aspects, the auxiliary medical device is a medical device with a first end anchored to the scaffold system and a second end configured to be engaged with tissue within a patient's body.

In some aspects, the delivery device includes a tubular elongate member configured to deliver the auxiliary medical device. In some aspects, the tubular elongate member is movable to move the scaffold system axially or rotationally.

In some aspects, the auxiliary medical device is a traction device, and movement of the delivery device adjusts the force vector the traction device may apply when engaged with tissue.

In accordance with various principles of the present disclosure, a method of delivering a scaffold system to a target site within a patient's body includes mounting the scaffold system on an elongate member to be movable with the elongate member; delivering the scaffold system with a distal end of the elongate member to the target site; shifting the configuration of the scaffold system to be released from the elongate member; and moving at least one of the scaffold system or the elongate member axially and rotationally with respect to the other of the scaffold system or elongate member.

In some aspects, the further includes anchoring the scaffold system with respect to tissue at the target site.

In some aspects, the method further includes expanding a first expandable element of the scaffold system to engage the elongate member to mount the scaffold system to the elongate member, and shifting the expandable element to an unexpanded configuration to release the scaffold system from the elongate member. In some aspects, the method further comprises expanding the first expandable element of the scaffold system or a second expandable element of the scaffold system to anchor the scaffold system with respect to tissue at the target site.

### BRIEF DESCRIPTION OF THE DRAWINGS

Non-limiting embodiments of the present disclosure are described by way of example with reference to the accompanying drawings, which are schematic and not intended to be drawn to scale. The accompanying drawings are provided for purposes of illustration only, and the dimensions, positions, order, and relative sizes reflected in the figures in the drawings may vary. For example, devices may be enlarged so that detail is discernable, but is intended to be scaled down in relation to, e.g., fit within a working channel of a delivery catheter or endoscope. In the figures, identical or nearly identical or equivalent elements are typically represented by the same reference characters, and similar elements are typically designated with similar reference numbers differing in increments of 1000, with redundant description omitted. For purposes of clarity and simplicity, not every element is labeled in every figure, nor is every element of each embodiment shown where illustration is not necessary to allow those of ordinary skill in the art to understand the disclosure.

The detailed description will be better understood in conjunction with the accompanying drawings, wherein like reference characters represent like elements, as follows:

FIG. 1 illustrates a perspective view of an embodiment of an auxiliary delivery system formed in accordance with aspects of the present disclosure, and illustrated positioned on an elongate member and within a schematic representation of a body lumen.

FIG. 2 illustrates a proximal perspective view of an auxiliary delivery system such as illustrated in FIG. 1.

FIG. 2A illustrates a cross-sectional view along line II-II of FIG. 2.

FIG. 3 illustrates a perspective view of an auxiliary delivery system such as in FIG. 1, positioned on an elongate member and being delivered to a target site within a body lumen.

FIG. 3A illustrates a cross-sectional view along line III-III of FIG. 3.

FIG. 4 illustrates a perspective view of the auxiliary delivery system of FIG. 3 deployed off the elongate member at a target site within a body lumen.

FIG. 4A illustrates a cross-sectional view along line IV-IV of FIG. 4.

FIG. 5 illustrates a perspective view of an auxiliary delivery system, similar to the auxiliary delivery system illustrated in FIG. 4, delivering an auxiliary medical device.

FIG. 6 illustrates a perspective view of an auxiliary delivery system, similar to the auxiliary delivery system illustrated in FIG. 4, delivering an auxiliary medical device via an auxiliary tubular elongate member.

FIG. 7 illustrates a perspective view similar to that of FIG. 6, but with a medical device delivered by the tubular elongate member delivering a cutting tool to cut tissue grasped and lifted by the auxiliary medical device delivered by the auxiliary tubular elongate member.

FIG. 8 illustrates a simplified elevational view of an example of an embodiment of an auxiliary delivery system formed in accordance with various principles of the present disclosure delivering an example of an embodiment of an auxiliary medical device.

FIG. 9 illustrates an auxiliary delivery system and auxiliary medical device such as illustrated in FIG. 8, but with the auxiliary medical device deployed and engaging tissue.

FIG. 10 illustrates a perspective view of an example of an embodiment of an auxiliary delivery system enhanced to inhibit migration once deployed.

FIG. 11 illustrates a cross-sectional view of an example of an embodiment of an auxiliary delivery system formed in accordance with various principles of the present disclosure.

FIG. 12 illustrates a perspective view of an example of an embodiment of an auxiliary delivery system formed in accordance with various principles of the present disclosure.

FIG. 13 illustrates a cross-sectional view along line XIII-XIII of FIG. 12.

FIG. 14 illustrates an elevational view of an auxiliary delivery system formed in accordance with various principles of the present disclosure, mounted for delivery on an elongate member.

FIG. 15 illustrates a perspective view of the auxiliary delivery system illustrated in FIG. 14, shifted into a configuration to be deployed from the elongate member.

FIG. 16 illustrates a distal perspective view of an auxiliary delivery system formed in accordance with various principles of the present disclosure.

FIG. 17 illustrates a proximal perspective view of an auxiliary delivery system as in FIG. 16.

FIG. 18 illustrates a proximal perspective view of a component of the auxiliary delivery system illustrated in FIG. 17.

FIG. 19 illustrates a perspective view of an auxiliary delivery system such as in FIG. 16 positioned on an elongate member and being delivered to a target site within a body lumen.

FIG. 19A illustrates a cross-sectional view along line IXX-IXX of FIG. 19.

FIG. 20 illustrates a perspective view of an auxiliary delivery system such as in FIG. 16 deployed within a body lumen.

FIG. 20A illustrates a cross-sectional view along line XX-XX of FIG. 20.

### DETAILED DESCRIPTION

The following detailed description should be read with reference to the drawings, which depict illustrative embodiments. It is to be understood that the disclosure is not limited to the particular embodiments described, as such may vary. All apparatuses and systems and methods discussed herein are examples of apparatuses and/or systems and/or methods implemented in accordance with one or more principles of this disclosure. Each example of an embodiment is provided by way of explanation and is not the only way to implement these principles but are merely examples. Thus, references to elements or structures or features in the drawings must be appreciated as references to examples of embodiments of the disclosure, and should not be understood as limiting the disclosure to the specific elements, structures, or features illustrated. It will be appreciated that the present disclosure is set forth in various levels of detail in this application. In certain instances, details that are not necessary for one of ordinary skill in the art to understand the disclosure, or that render other details difficult to perceive may have been omitted. The terminology used herein is for the purpose of describing particular embodiments only, and is not intended to be limiting beyond the scope of the appended claims. Unless defined otherwise, technical terms used herein are to be understood as commonly understood by one of ordinary skill in the art to which the disclosure belongs. All of the devices and/or methods disclosed and claimed herein can be made and executed without undue experimentation in light of the present disclosure.

As used herein, “proximal” refers to the direction or location closest to the user (medical professional or clinician or technician or operator or physician, etc., such terms being used interchangeably herein without intent to limit, and including automated controller systems or otherwise), etc., such as when using a device (e.g., introducing the device into a patient, or during implantation, positioning, or delivery), and/or closest to a delivery device, and “distal” refers to the direction or location furthest from the user, such as when using the device (e.g., introducing the device into a patient, or during implantation, positioning, or delivery), and/or closest to a delivery device. “Longitudinal” means extending along the longer or larger dimension of an element. A “longitudinal axis” extends along the longitudinal extent of an element, though is not necessarily straight and does not necessarily maintain a fixed configuration if the element flexes or bends, and “axial” generally refers to along the longitudinal axis. However, it will be appreciated that reference to axial or longitudinal movement with respect to the above-described systems or elements thereof need not be strictly limited to axial and/or longitudinal movements along a longitudinal axis or central axis of the referenced elements. “Central” means at least generally bisecting a center point and/or generally equidistant from a periphery or boundary, and a “central axis” means, with respect to an opening, a line that at least generally bisects a center point of the opening, extending longitudinally along the length of the opening when the opening comprises, for example, a tubular element, a strut, a channel, a cavity, or a bore. As used herein, a “lumen” or “channel” or “bore” or “passage” is not limited to a circular cross-section. As used herein, a “free end” of an element is a terminal end at which such element does not extend beyond. It will be appreciated that terms such as at or on or adjacent or along an end may be used interchangeably herein without intent to limit unless otherwise stated, and are intended to indicate a general relative spatial relation rather than a precisely limited location. Finally, reference to “at” a location or site is intended to include at and/or about the vicinity of (e.g., along, adjacent, proximate, etc.) such location or site. As understood herein, corresponding is intended to convey a relationship between components, parts, elements, etc., configured to interact with or to have another intended relationship with one another.

Generally, when target tissue within or in the area of a target site within a patient's body is to be removed, or needs to be repaired, it is desirable to do so using a minimally-invasive procedure. In contrast with open surgery, a medical device is advanced into a patient's body through a natural orifice (or small incision, if necessary) and navigated within the body, such as through an internal body passage) to a target site. It will be appreciated that terms such as devices, instruments, tools, components, etc., may be used interchangeably herein without intent to limit unless otherwise indicated. It will further be appreciated that terms such as target site, target area, target tissue site, target tissue area, target area of tissue, target treatment area, treatment area, target treatment site, treatment site, etc., may be used interchangeably herein, without intent to limit, to refer to an area or region of tissue within which a target tissue is located. The term target tissue (and other variations thereof, such as treatment tissue) is used herein to refer to the tissue with respect to which a procedure is to be performed or which is to be treated or otherwise operated on or affected by the devices and/or systems and/or methods disclosed herein. Thus, the target site may be understood as an area or region extending outwardly from or around or surrounding the target tissue (specific tissue in the target tissue area), such as the region a medical professional would consider a working area around the target tissue for performing a procedure with respect to the target tissue.

Typically, an elongate member is used in minimally-invasive medical procedures to deliver a medical device to a target site. Typically, the elongate member is sufficiently flexible to navigate through tortuous body passages, particularly if inserted transluminally. It is generally beneficial for the delivery device to be steerable, and the delivery device may have different areas of different flexibility or stiffness to promote steerability. The elongate member may include a shaft with a medical device mounted or otherwise coupled to a distal end thereof. In some aspects, the elongate member is tubular and defines a lumen through which a medical device is advanced to a target site. A tubular elongate member used as a delivery device may be in the form of a catheter, sheath, tube, cannula, etc. (such terms being used interchangeably herein without intent to limit) or other configuration of an introducer. A tubular elongate member may or may not have a medical device mounted or otherwise coupled to a distal end thereof. In some aspects, it may be advantageous to deliver a medical device through a delivery device in the form of a tubular elongate member to protect or shield the passage through which the medical device is delivered (e.g., body passage or otherwise) against adverse interactions (e.g., catching, scratching, and/or otherwise) by the medical device delivered therethrough. Additional overtubes or sheaths may be provided as desired or as necessary to reduce friction or interference of the exterior of any elements with another element.

In addition to the above-described elongate members, various medical devices, such as medical scopes (e.g., endoscopes, arthroscopes, bronchoscopes, colonoscopes, cystoscopes, duodenoscopes, gastroscopes, hysteroscopes, laparoscopes, ureteroscopes, etc.), include an elongate member for insertion into a body passageway or cavity to enable a medical professional to deliver a medical device to a target site within a patient's body to perform minimally-invasive surgical procedures at the target site internal to the patient's body. Medical scopes typically have a proximal end (typically with a control handle) that remains external to the patient, and a distally extending flexible elongate member configured for insertion into the patient. The distally-extending flexible elongate member is generally tubular, and may be known as an insertion tube (and may referenced herein as such for the sake of convenience and without intent to limit). The distally-extending flexible elongate member of the medical scope is inserted into a body cavity or passage of the patient, and is navigated within the patient to an internal treatment site. In general, the distally-extending flexible elongate member of the medical scope has one or more working channels extending longitudinally therethrough through which a medical device may be advanced or retracted. The distally-extending flexible elongate member of the medical scope may also be equipped with one or more accessories such as, for example, a miniature viewing device (optical component, such as a camera), an illumination device (e.g., an LED or optical fiber), lumens for suction, lumens for inflation/irrigation mediums, etc. The distally-extending flexible elongate members of most medical scopes generally have a limited number of working channels, and the outer diameter of such devices generally is limited by the size of the body passage through which the device is to be navigated.

For purposes of the present disclosure, an elongate member with a medical device at a distal end thereof, or a tubular elongate member through which another medical device may be delivered to a target site, or the distally-extending flexible elongate member of a medical scope, or another form of an elongate member may be considered herein to be a delivery device, or, more generally an “elongate member” with which systems, devices, and methods of the present disclosure may be used. For the sake of convenience, reference is made herein simply to an elongate member. It should be appreciated that such elongate member may have a medical device mounted or coupled to a distal end thereof (e.g., as an integral device), or may be a tubular elongate member with a medical device extended therethrough. The elongate member may be any suitable size, cross-sectional shape or area, and/or configuration permitting introduction and passage of devices or instruments to the distal end of the elongate member, such as for passage and introduction of medical instruments to a target tissue site. Typically, the elongate member has at least a distal portion which is elongate and flexible to be able to be navigated through passages within a patient's body which may be curved and/or tortuous, with a proximal portion extending proximally (out of the patient's body or coupled with a control handle outside the patient's body) to be controlled (maneuvered, or one or more functions thereof, such as camera, irrigation, suction, light, etc., actuated) by a medical professional. An auxiliary delivery system formed in accordance with various principles of the present disclosure may be delivered with (e.g., over) such an elongate member to a target site.

In accordance with various principles of the present disclosure, an auxiliary delivery system is delivered along with (movable with) an elongate member configured to deliver a medical device to a target site within a patient's body. In some aspects, the auxiliary delivery system is mounted along a distal end of the elongate member for delivery with the distal end of the elongate member to a target site within a patient's body. In some aspects, the auxiliary delivery system may be considered to have a delivery configuration in which the auxiliary delivery system engages the elongate member to move therewith for delivery to a target site. The auxiliary delivery system is separable from the elongate member once delivered to the target site for independent axial and rotational movement of the auxiliary delivery system and the elongate member (with movement of one of the auxiliary delivery system or the elongate member not affecting the other of the auxiliary delivery system or elongate member). In some aspects, the auxiliary delivery system may be considered to have a deployed configuration in which the auxiliary delivery system is separated from the elongate member such that the auxiliary delivery system and the elongate member are independently axially and rotationally movable with respect to each other. In some aspects, the auxiliary delivery system may have a further deployed configuration in which the auxiliary delivery system engages tissue at the target site to anchor the auxiliary delivery system with respect to the target site. In some aspects, the target site is within a body lumen and the auxiliary delivery system expands radially outwardly (and may have a generally cylindrical/tubular exterior shape) to engage the tubular inner walls of the body lumen. It will be appreciated that reference to a body lumen includes naturally-existing lumens or passages (e.g., the colon) as well as medically-created lumens (e.g., a lumen or passage created with the use of a medical instrument, and not existing without medical intervention) or otherwise. In some aspects, the auxiliary delivery system includes actuators to shift the auxiliary delivery system among its various configurations.

In some aspects, an auxiliary delivery system formed in accordance with various principles of the present disclosure includes a scaffold system which shifts the configuration of the auxiliary delivery system (or at least a portion thereof) between a delivery configuration and one or more deployed configurations. In some aspects, the scaffold system includes a first scaffold member positioned, configured, and/or adapted to anchor the auxiliary delivery system with respect to an elongate member for delivery therewith to a target site when the first scaffold member is in a delivery configuration. The first scaffold member may be shifted to a deployed configuration for release from the elongate member. Alternatively or additionally, the scaffold system includes a second scaffold member positioned, configured, and/or adapted to stabilize the auxiliary delivery system with respect to tissue in the vicinity of the target site when the second scaffold member is in a deployed configuration. In some aspects, stabilizing of the auxiliary delivery system stabilizes an auxiliary delivery device of the system. In some aspects, the scaffold system is shiftable from a compact delivery configuration, such as delivered and/or coupled outside the elongate member, to an expanded configuration, such as spaced apart from the elongate member. In some aspects, the delivery configuration minimally affects the outer profile/outer diameter of the elongate member. In some aspects, in the expanded configuration, the scaffold system anchors the auxiliary delivery system with respect to tissue. In some aspects, the scaffold system includes a first scaffold member and/or a second scaffold member which is selectively shiftable between an expanded and an unexpanded configuration.

In some aspects, the auxiliary delivery system is configured to deliver an auxiliary medical device (in addition to a medical device delivered by the elongate member) to the target site. In some aspects, the auxiliary delivery system of the present disclosure is movable independently of the elongate member such as to allow axial (e.g., longitudinal translation) and/or rotational movement with respect to the elongate member. The degrees of freedom of movement which the auxiliary delivery system of the present disclosure and an elongate member have relative to each other allow for greater degrees of freedom of movement of an auxiliary medical device delivered by the auxiliary delivery system of the present disclosure. The present disclosure describes various configurations of auxiliary delivery systems formed in accordance with various principles of the present disclosure.

In some aspects, an auxiliary delivery system formed in accordance with various principles of the present disclosure provides an auxiliary elongate member in addition to a elongate member with which the auxiliary delivery system is delivered. The auxiliary elongate member may be coupled to another component of the auxiliary delivery system (e.g., a scaffold system) in any of a variety of manners including, without limitation, adhering (e.g., with adhesive), bonding, welding, tying, etc. In some aspects, the auxiliary elongate member defines a working channel or lumen therethrough. As may be appreciated, such auxiliary working channel allows for delivery of additional/auxiliary medical devices while a medical device is delivered through the working channel of the tubular elongate member. The auxiliary lumen may be used as an auxiliary delivery device independently of the tubular elongate member. It will be appreciated that the auxiliary elongate member may be particularly beneficial/useful in conjunction with a tubular elongate member which only has a single delivery lumen (e.g., an endoscope with a single working channel), or a lumen of limited diameter. The auxiliary elongate member may have a significantly smaller outer diameter so as not to interfere with advancing of the tubular elongate member within a patient's body to a target site. It will be appreciated that the addition of an auxiliary lumen along the exterior of the elongate member in accordance with various principles of the present disclosure increases the overall diameter and cross-sectional area of the system less than if the elongate member were modified to include another working channel therethrough. Moreover, the addition of another working channel to the elongate member might impact the flexibility of the elongate member as it is navigated within the body (such as through tortuous body passages), whereas an auxiliary lumen dimensioned for a filament or a single working instrument may be generally more flexible than the elongate member. Moreover, the auxiliary elongate member does not impact the flexibility of an existing elongate member because the auxiliary elongate member is not permanently coupled with the elongate member, and may be coupled with the elongate member along only a limited extent (e.g., only along the distal end of the elongate member) for delivery with the elongate member. Alternatively or additionally, the auxiliary elongate member and the auxiliary delivery system may be movable independently of the tubular elongate member, such as described above, and therefore may allow the additional medical device to move independently of the tubular elongate member, its working channel, and/or any medical device delivered by the tubular elongate member. As may be appreciated, auxiliary delivery systems formed in accordance with various principles of the present disclosure thereby provide greater flexibility to the medical professional in the use of medical devices and the operation of such devices with respect to tissue at a target site.

In some aspects, the auxiliary delivery system delivers a traction device. The traction device may include a tether, traction band, suture, string, cord, wire, filament, etc., whether clastic or inelastic, which is coupled to tissue to apply traction to the tissue, such as to lift cut tissue from surrounding tissue and thus away from a field of view and/or a cutting tool. The traction device may be delivered through an auxiliary delivery device delivered by the auxiliary delivery system, or may be mounted on an exterior of a component of the auxiliary delivery system. Because the auxiliary delivery system is movable independently of the elongate member, movement of the traction device (e.g., to apply traction, adjust traction, adjust a force vector applied by the tether, etc.) may be effected independently of the elongate member and any medical device delivered by the elongate member, and movement of the elongate member does not affect the traction device.

It will be appreciated that different sizes and configurations and arrangements of components of an auxiliary delivery system formed in accordance with various principles of the present disclosure may be selected or set depending on the procedure, the nature of the target site (e.g., location, area/size, type of tissue, etc.), preferences of the medical professional. Various embodiments of auxiliary delivery systems, and devices, systems, and methods for delivering and/or maneuvering a medical device independently of another delivery device and another medical device (such as delivered by the other delivery device) will now be described with reference to examples illustrated in the accompanying drawings. Reference in this specification to “one embodiment,” “an embodiment,” “some embodiments”, “other embodiments”, etc. indicates that one or more particular features, structures, concepts, and/or characteristics in accordance with principles of the present disclosure may be included in connection with the embodiment. However, such references do not necessarily mean that all embodiments include the particular features, structures, concepts, and/or characteristics, or that an embodiment includes all features, structures, concepts, and/or characteristics. Some embodiments may include one or more such features, structures, concepts, and/or characteristics, in various combinations thereof. It should be understood that one or more of the features, structures, concepts, and/or characteristics described with reference to one embodiment can be combined with one or more of the features, structures, concepts, and/or characteristics of any of the other embodiments provided herein. That is, any of the features, structures, concepts, and/or characteristics described herein can be mixed and matched to create hybrid embodiments, and such hybrid embodiment are within the scope of the present disclosure. Moreover, references to “one embodiment,” “an embodiment,” “some embodiments”, “other embodiments”, etc. in various places in the specification are not necessarily all referring to the same embodiment, nor are separate or alternative embodiments necessarily mutually exclusive of other embodiments. It should further be understood that various features, structures, concepts, and/or characteristics of disclosed embodiments are independent of and separate from one another, and may be used or present individually or in various combinations with one another to create alternative embodiments which are considered part of the present disclosure. Therefore, the present disclosure is not limited to only the embodiments specifically described herein, as it would be too cumbersome to describe all of the numerous possible combinations and subcombinations of features, structures, concepts, and/or characteristics, and the examples of embodiments disclosed herein are not intended as limiting the broader aspects of the present disclosure. It should be appreciated that various dimensions provided herein are examples and one of ordinary skill in the art can readily determine the standard deviations and appropriate ranges of acceptable variations therefrom which are covered by the present disclosure and any claims associated therewith. The following description is of illustrative examples of embodiments only, and is not intended as limiting the broader aspects of the present disclosure.

It will be appreciated that common features are identified by common reference elements and, for the sake of brevity and convenience, and without intent to limit, the descriptions of the common features are generally not repeated. For purposes of clarity, not all components having the same reference number are numbered. Moreover, a group of similar elements may be indicated by a number and letter, and reference may be made generally to one or such elements or such elements as a group by the number alone (without including the letters associated with each similar element). It will be appreciated that, in the following description, elements or components similar among the various illustrated embodiments with reference numbers greater than 1000 are generally designated with the same reference numbers increased by a multiple of 1000 and redundant description is generally omitted for the sake of brevity. Moreover, certain features in one embodiment may be used across different embodiments and are not necessarily individually labeled when appearing in different embodiments.

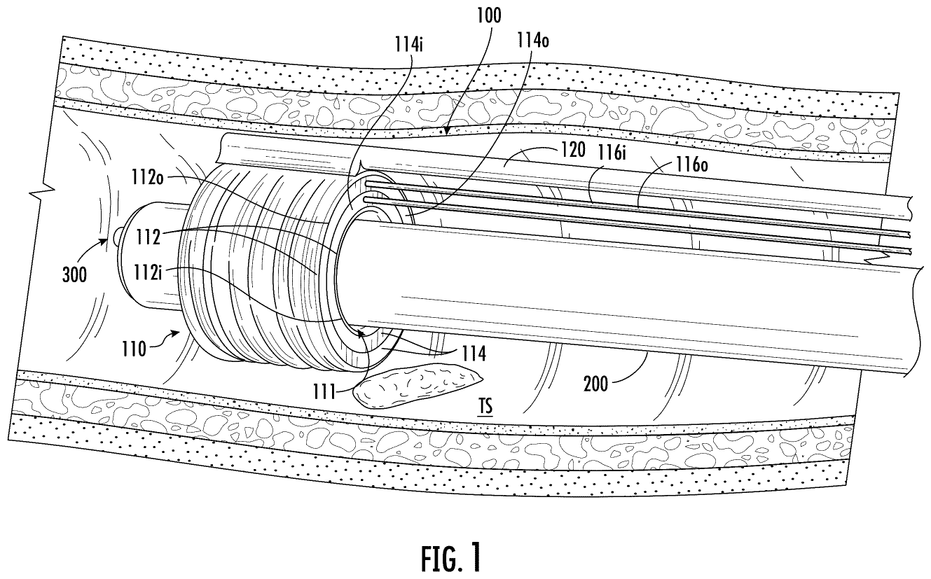

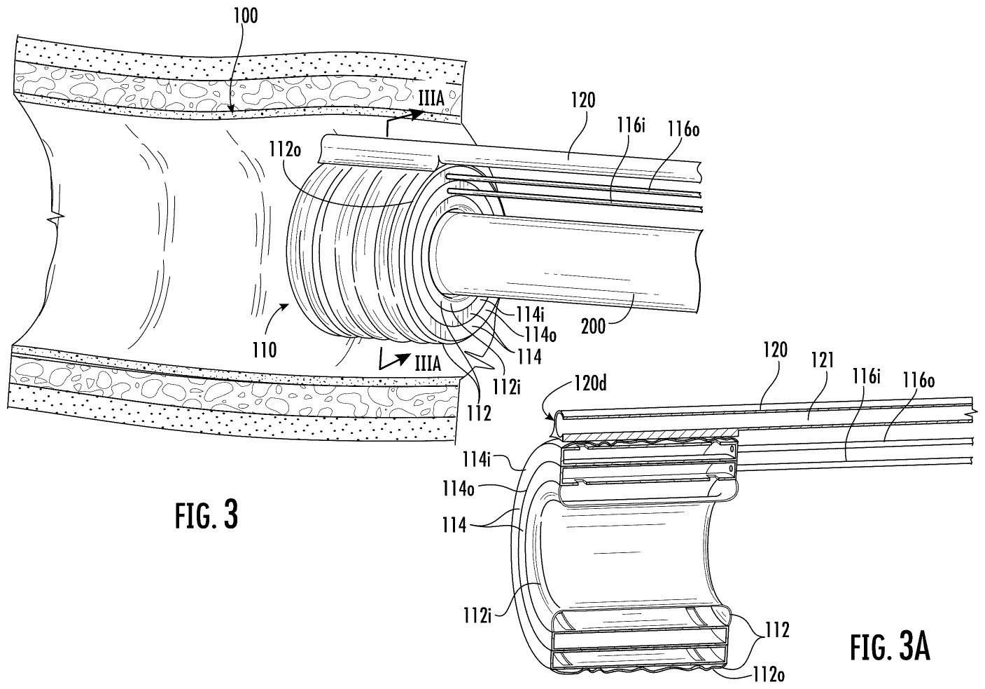

Turning now to the drawings, an example of an embodiment of an auxiliary delivery system 100 formed in accordance with various principles of the present disclosure is illustrated in FIG. 1. The illustrated auxiliary delivery system 100 includes a scaffold system 110 configured to be mounted with respect to an elongate member (such as illustrated in FIG. 3) for delivery to a target site TS. In some aspects, an auxiliary elongate member 120 is operably associated with the scaffold system 110 to be delivered together with the scaffold system 110 and elongate member to a target site TS. In some aspects, the elongate member is a tubular elongate member defining a lumen or working channel (such as known in the art and thus not warranting illustration for an understanding thereof) through which a medical device is deliverable to a target site TS. For instance, in some aspects, the elongate member may be an endoscope with a working channel for a medical instrument to be advanced therethrough to a target site TS. In accordance with various principles of the present disclosure, the scaffold system 110 defines a lumen 111 therethrough through which the elongate member is extended. In such configuration, the scaffold system 110 is mounted over and generally surrounding the elongate member for delivery therewith to a target site TS. Because the entire length of the elongate member typically is not delivered to the target site TS, and typically at least the distal end of the elongate member is delivered to the target site TS, the scaffold system 110 is typically mounted along the distal end of the elongate member.

In accordance with various principles of the present disclosure, the scaffold system 110 is configured to be releasably mounted on the elongate member 200. The scaffold system 110 thus is operably associated with the elongate member 200 during delivery of the elongate member 200 to a target site TS to be advanced therewith to a target site TS. The scaffold system 110 is releasable from the elongate member 200, such as once delivered to the target site TS, to be deployed separately (e.g., spaced apart) from the elongate member 200 at the target site TS. Once deployed from the elongate member 200, the elongate member 200 and the scaffold system 110 are independently movable (such as axially or rotationally movable) with respect to each other. For instance, the elongate member 200 is independently movable (such as axially and rotationally) with respect to the scaffold system 110 once the scaffold system 110 has been deployed from the elongate member 200. In some aspects, the scaffold system 110 may be moved once deployed from the elongate member 200, and, because the scaffold system 110 is no longer mounted on the elongate member 200, the scaffold system 110 may be moved independently (such as axially and rotationally) with respect to the elongate member 200. Moreover, the scaffold system 110 is reconfigurable to engage tissue at the target site TS to anchor the scaffold system 110 with respect to the target site TS once released from the elongate member 200 (e.g., a further deployed configuration engaged and/or anchored with respect to tissue).

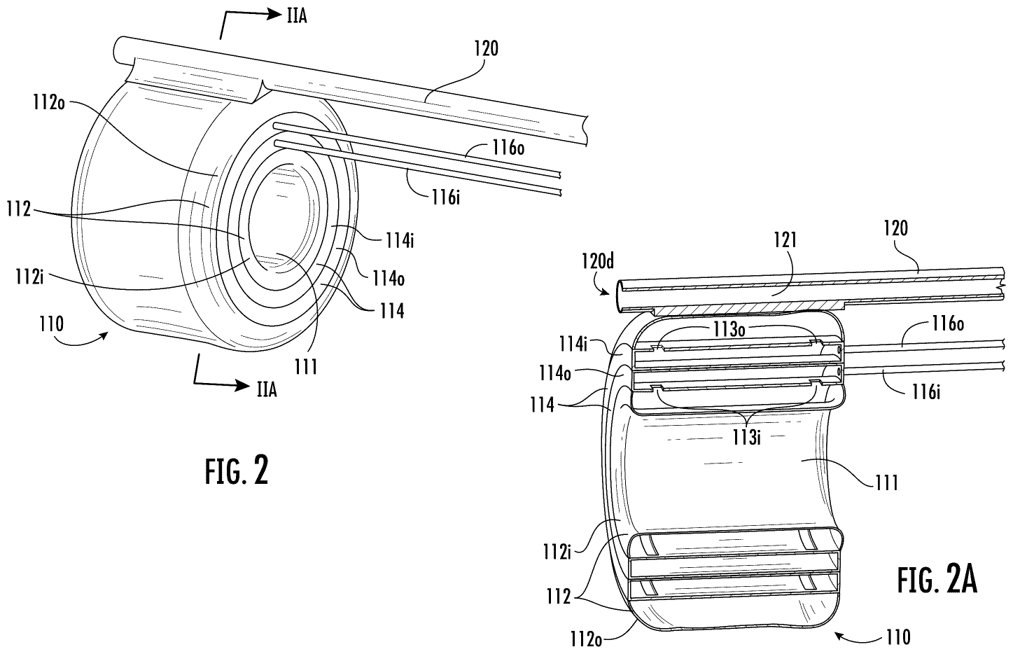

The example of an embodiment of a scaffold system 110 formed in accordance with various principles of the present disclosure and illustrated in FIG. 1, FIG. 2, FIG. 2A, FIG. 3, and FIG. 3A has one or more expandable elements 112 configured to mount the scaffold system 110 with respect to the elongate member 200 and/or to anchor the scaffold system 110 with respect to tissue such as tissue at a target site TS. In some aspects, the scaffold system 110 includes a first expandable element 112i and a second expandable element 112o which are separately and independently actuatable to expand or to contract. In some aspects, the scaffold system 110 includes a support structure 114 for the first expandable element 112i and/or the second expandable element 112o. In some aspects, the support structure 114 includes a first support structure 114i on which the first expandable element 112i is supported, and a second support structure 114o on which the second expandable element 112o is supported. Each of the expandable elements 112 and support structures 114 may be generally tubular to define the lumen 111 of the scaffold system 110 through which the elongate member 200 extends. Alternatively or additionally, the expandable elements 112 may be mounted concentrically, or otherwise one on top of the other, so that, in the illustrated example of an embodiment, the first expandable element 112i is an inner expandable element 112i mounted on an inner support structure 114i and the second expandable element 112o is an outer expandable element 112o supported on an outer support structure 114o. In such configuration, the inner expandable element 112i may contact the exterior of the elongate member 200 and the outer expandable element 112o may contact tissue when the scaffold system 110 is deployed at a target site TS. In the illustrated example of an embodiment, the first expandable element 112i is an innermost expandable element, and the second expandable element 112o is an outermost expandable element. It will be appreciated that additional expandable elements may be positioned between the innermost expandable element and the outermost expandable element, the present disclosure not being limited in this regard.

In the example of an embodiment illustrated in FIG. 1, each of the expandable elements 112 is an inflatable member which may be selectively shifted between an expanded and unexpanded configuration (independently of each other). In some aspects, the actuator element used to shift the expandable elements 112 between configurations is an inflation lumen configured to deliver or withdraw an inflation medium (e.g., air, saline, etc.) to/from the expandable elements 112 as needed during a procedure. As may be better appreciated with reference to FIG. 2 and the cross-sectional view thereof (along line IIA-IIA) in FIG. 2A, an inflation lumen 116i, 116o may fluidly couple a respective expandable elements 112i, 112o with an inflation medium source (not shown, but which may be any known inflation medium source known or heretofore known to those of ordinary skill in the art) to inflate or deflate the expandable elements 112i, 112o such as in a manner known to those of ordinary skill in the art. As may be appreciated with reference to FIG. 2A, the inflation lumens 116i, 116o may be respectively coupled to and in fluid communication with the interiors of hollow support structures 114i, 114o which respectively support the expandable elements 112i, 112o. One or more fluid communication apertures 113i may fluidly communicate the interior of the inner support structure 114i with the interior of the inner expandable elements 112i to fluidly communicate the inner inflation lumen 116i with the inner expandable element 112i. Similarly, one or more fluid communication apertures 113o may fluidly communicate the interior of the outer support structure 114o with the interior of the outer expandable elements 112o to fluidly communicate the outer inflation lumen 116o with the outer expandable element 112o.

In accordance with various principles of the present disclosure, the inner expandable element 112i and the outer expandable element 112o are independently expandable. For instance, the inner expandable element 112i and the outer expandable element 112o may be fluidly isolated, or otherwise not in fluid communication with each other. The independent expandability of the expandable elements 112i, 112o allows for the expandable elements 112i, 112o to be expanded at different times, rates, degrees, etc., such as for different purposes. For instance, as may be appreciated with reference to FIG. 3 and the cross-sectional view along line IIIA-IIIA thereof illustrated in FIG. 3A, the inner expandable element 112i may be expanded to reduce the diameter/cross-sectional area of the lumen 111 within the scaffold system 110. Such delivery configuration allows the scaffold system 110 to firmly engage, grab, purchase, etc., (such terms being used interchangeably herein without intent to limit, and including, without limitation, interference or compression fits with) the exterior of the elongate member 200 extending through the lumen 111 to maintain engagement with the elongate member 200 for delivery therewith to a target site TS. The outer expandable element 112o may remain substantially unexpanded as illustrated. In such collapsed delivery configuration, the thickness of the outer expandable element 112o (in a generally radial direction) has minimal impact on the overall dimensions of the auxiliary delivery system 100, and minimal impact on the navigability of the auxiliary delivery system 100.

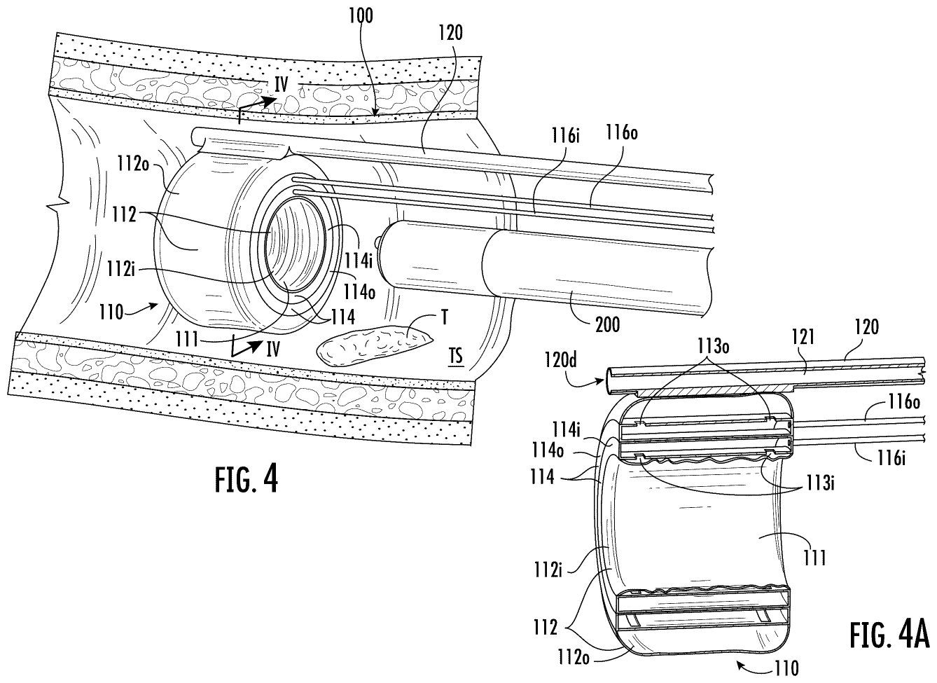

Once the auxiliary delivery system 100 and elongate member 200 have reached the target site TS, the auxiliary delivery system 100 is separable from the elongate member 200, as illustrated in FIG. 4. More particularly, the inner expandable element 112i is shifted to an unexpanded deployed configuration (e.g., is deflated) to increase the diameter and overall cross-sectional area of the lumen 111 of the scaffold system 110 so that the scaffold system 110 may be moved distally off of the elongate member 200. Because the scaffold system 110 is operably engaged with the elongate member 200 solely by the expansion of the inner expandable element 112i, reduction of the size of the inner expandable element 112i with consequent increase in the inner diameter defined by the inner expandable element 112i (which may be considered to define at least a portion of the lumen 111 of the scaffold system 110) allows full separation of the scaffold system 110 from the elongate member 200. The scaffold system 110 and the elongate member 200 are thus capable of fully independent movement, axially and rotationally, with respect to each other once the scaffold system 110 is no longer operably associated with the elongate member 200. The elongate member 200 may be moved axially and/or rotationally independently of the scaffold system 110 once the scaffold system 110 has been deployed, without affecting the position, orientation, etc., of the scaffold system 110. In some aspects, the scaffold system 110 may be advanced distally from and off of the elongate member 200 to a desired location and/or may be rotated with respect to the elongate member 200, such as to adjust the position of the scaffold system 110 and/or the auxiliary elongate member 120 relative to one or more of the components of the elongate member 200 (e.g., the working channel, the camera, the light, the irrigation/suction channel, etc., of an elongate member 200 in the form of an endoscope). The outer expandable element 112o may then be expanded (e.g., inflated) to anchor or otherwise secure the position of the scaffold system 110 with respect to the target site TS. In some aspects, the auxiliary elongate member 120 is coupled with the scaffold system 110 and controllable by a medical professional to move the scaffold system 110 with respect to the elongate member 200. Any of a variety of control handles or the like may be used along the proximal end of the auxiliary elongate member 120 to control movement of the auxiliary elongate member 120 to thereby control movement of the scaffold system 110, the present disclosure not being limited in this regard.

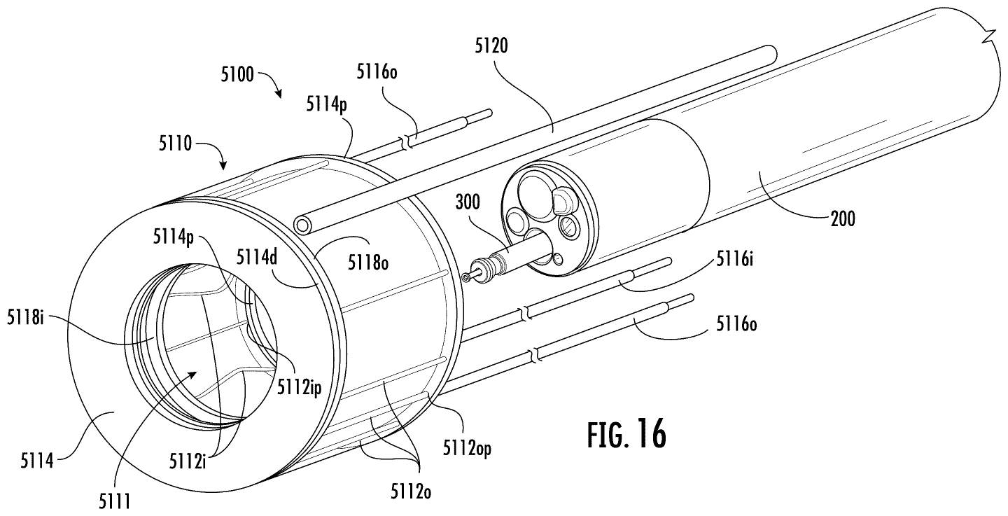

In accordance with various principles of the present disclosure, the auxiliary delivery system 100 may be configured to deliver a medical device to the target site TS. In embodiments in which the elongate member 200 over which the scaffold system 110 is delivered is a tubular elongate member 200 with a working channel (see, e.g., FIG. 16), the elongate member 200 may be considered a delivery device for a medical instrument, and the auxiliary delivery system 100 may be considered to provide an auxiliary delivery device in conjunction with the elongate member 200.

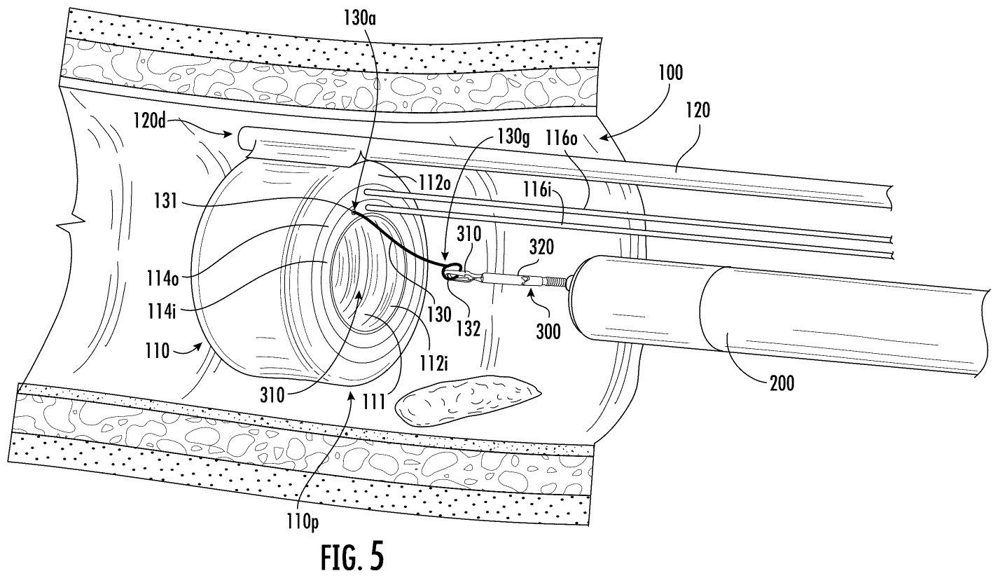

In some aspects, the scaffold system 110 of the auxiliary delivery system 100 may be considered to be an auxiliary delivery device of the auxiliary delivery system 100. For instance, as illustrated in FIG. 5, the scaffold system 110 may be configured to deliver a traction device 130, and the elongate member 200 may be configured to deliver a medical device 300 with an engagement member 310 configured to grasp the traction device 130, such as along an end 130g positioned for grasping. In some aspects, a feature may be associated with the traction device 130 and/or the scaffold system 110 to hold the end 130g in a desired position, such as during delivery. For instance, optional features include, without limitation, a feature may be provided on a component of the scaffold system 110 (e.g., on one of the expandable elements 112i, 112o) to hold a portion of the traction device 130; or a portion of the traction device 130 may be held with respect to the scaffold system 110 with an adhesive, peelable glue, etc.; or the traction device 130 may be housed within a structure (e.g., a cavity) associated with and/or within the scaffold system 110 and withdrawn therefrom (e.g., by pulling on a portion of the end 130g) once ready for use. In some aspects, the end 130g may include a loop 132 to more readily enable the engagement member 310 to grasp the traction device 130. An end 130a of the traction device 130 may be coupled to the scaffold system 110 at an anchor point 131 on the scaffold system 110 (on the support structure 114 and/or an expandable element 112i, 112o), such as along the proximal end 110p of the scaffold system 110. It will be appreciated that the traction device 130 may be anchored to the support structure 114 or to at least one of the expandable elements 112i, 112o of the scaffold system 110, with various accompanying benefits. For instance, the support structure 114 may provide stability to the anchoring of the traction device 130. In contrast, if the traction device 130 is anchored to an expandable element 112, then expansion of the expandable element 112 may provide further adjustability/manipulability of the position of the traction device 130. For instance, the traction applied by the traction device 130 (e.g., the magnitude of the traction) can be manipulated by adjusting the expansion (e.g., outer pressure) of the outer expandable element 112o by moving the distance of the anchor point 131 of the traction device 130 relative to the target tissue T (e.g., to stretch the traction device 130 and thereby increase traction the traction device 130 applies to the target tissue T).

In some aspects, the engagement member 310 is configured to operably couple the traction device 130 to target tissue T at the target site TS. The engagement member 310 may be in the form of a grasper, forceps, clamp, etc., actuatable (e.g., by moving a pair of jaws thereof apart or together) to grasp a free end of the traction device (i.e., an end not coupled to any component of the auxiliary delivery system) and/or tissue. In some aspects, the engagement member 310 is configured to engage tissue to operably couple the traction device 130 to the target tissue T. In some aspects, the engagement member 310 is releasable from the shaft 320 on which it is delivered to remain in place at the target site TS (holding an end of the traction device 130 to the target tissue T) while the shaft 320 is retracted and removed from the target site TS. Examples of engagement members in the form of a clip may include, but are not limited to, those described in U.S. Pat. No. 7,494,461, issued Feb. 24, 2009, and titled “Through The Scope Tension Member Release Clip”; U.S. Pat. No. 8,062,311, issued Nov. 22, 2011, and titled “Endoscopic Hemostatic Clipping Apparatus”; U.S. Pat. No. 8,080,021, issued Dec. 20, 2011, and titled “Multiple Clip Deployment Magazine”; and U.S. Patent Application Publication 2009/0187198, filed Dec. 15, 2008, and titled “Resolution Clip”, all of which are hereby incorporated by reference herein in their entirety and for all purposes. It will be appreciated that the traction device 130 is only one example of a medical device deliverable by the auxiliary delivery system 100 of the present disclosure.

It will be appreciated by those of ordinary skill in the art that the maneuverability of the scaffold system 110 and the elongate member 200 independently of each other allows the scaffold system 110 to be rotated or axially translated (advanced, retracted, etc.) with respect to the target tissue T and/or the elongate member 200 to position the scaffold system 110 at an optimal location, position, orientation, etc., with respect to the target tissue T and/or the elongate member 200 while the elongate member 200 may be moved independently of such location, position, orientation, etc. For instance, the anchor point 131 of a traction device 130 delivered by the scaffold system 110 may be positioned with respect to target tissue T to apply a desired force vector with respect to the target tissue T. More particularly, the traction device 130 may be coupled to the target tissue T to apply traction to the target tissue T, such as during cutting of the tissue (e.g., during a tissue resection or tissue dissection procedure), to lift the tissue, such as (without limitation) away from a cutting device delivered by the elongate member 200 and/or a visualization device of the elongate member 200. The force vector applied by the traction device 130 may be adjusted by adjusting the position of the scaffold system 110 without affecting the position or operation of other medical instruments used during the procedure and operably associated with the elongate member 200 (such as delivered through the working channel of the elongate member 200, or formed with the elongate member 200, such as a visualization device). Moreover, the auxiliary delivery system 100 of the present disclosure allows for maneuverability and positioning of the scaffold system 110 during a procedure without interfering with the elongate member 200 or any medical instruments extended therethrough for use during the procedure. Likewise, movements of the elongate member 200 and any medical instruments associated therewith do not affect the position or other aspects of the auxiliary delivery system 100.

Alternatively or additionally, in accordance with various principles of the present disclosure, the auxiliary elongate member 120 may be a tubular auxiliary elongate member 120 (such as illustrated in FIG. 2A, FIG. 3A, and FIG. 4A) configured to deliver a medical device to the target site TS. It will be appreciated that despite the various benefits of the lumen 121 of a tubular auxiliary elongate member 120, as described below, if the auxiliary elongate member 120 does not include a lumen therethrough, the auxiliary elongate member 120 may nonetheless still be beneficial with assisting maneuvering of the scaffold system 110 with respect to and independently of the elongate member 200 and/or otherwise delivery auxiliary medical devices.

A tubular auxiliary elongate member 120 may deliver a medical device in addition to any medical instrument deliverable with the elongate member 200, and thus may be considered an auxiliary delivery device or auxiliary elongate member. In some aspects, the auxiliary elongate member 120 may be considered an auxiliary delivery device of the auxiliary delivery system 100 delivered with/by the scaffold system 110. An auxiliary medical device 140 (auxiliary or additional to any medical devices delivered by the elongate member 200) may thus be delivered to the target site TS by the auxiliary elongate member 120, such as illustrated in

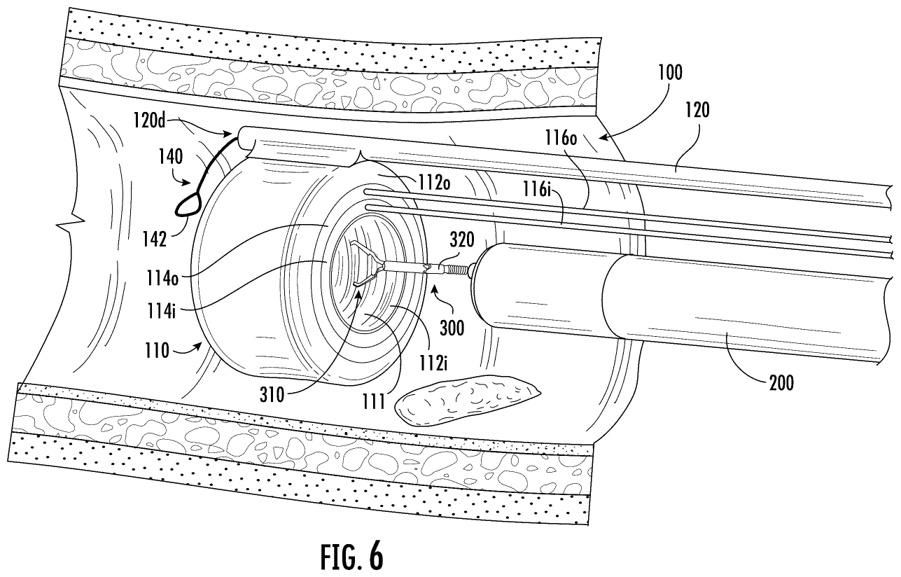

FIG. 6. The auxiliary medical device 140 may exit the lumen 121 of the auxiliary elongate member 120 via the distal end 120d of the auxiliary elongate member 120. In some aspects, an auxiliary medical device 140 exiting the distal end 120d of the auxiliary elongate member 120 exits the auxiliary elongate member 120 distal to the scaffold system 110, such as illustrated in FIG. 6. The auxiliary medical device 140 may be stowed within the lumen 121 of the auxiliary elongate member 120 during delivery of the auxiliary delivery system 100 to the target site TS and extended distally out of the lumen 121 once the auxiliary delivery system 100 has been delivered to the target site TS (e.g., once the auxiliary delivery system 100 has been deployed at the target site TS). Alternatively, a distal end of the auxiliary medical device 140 may extend within the lumen 111 of the scaffold system 110 during deliver of the auxiliary delivery system 100, held between the scaffold system 110 and the exterior of the elongate member 200. In some aspects, a medical device 300 advanced through the elongate member 200 may be extended out of the elongate member 200 to interact with the auxiliary medical device 140, such as illustrated in FIG. 6.

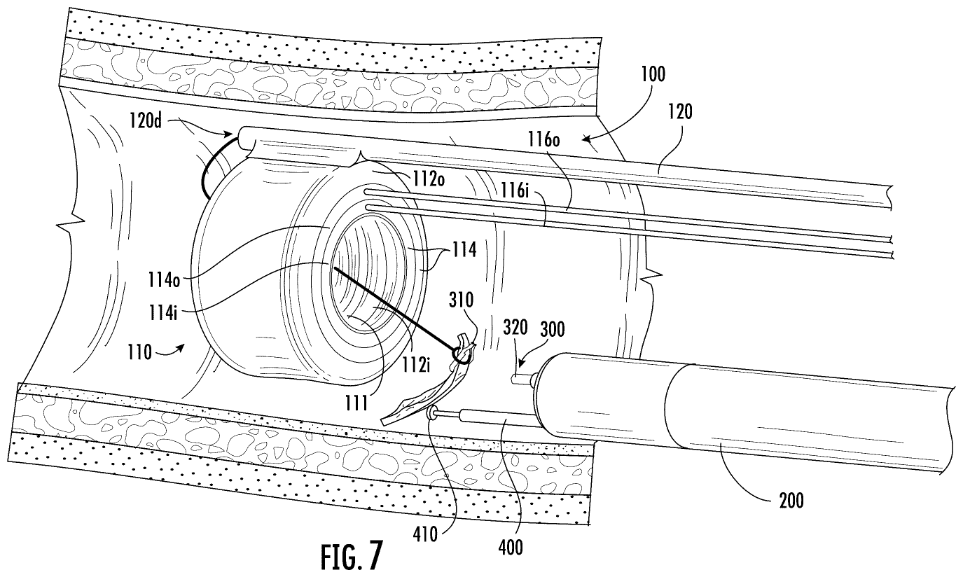

In the example of an embodiment illustrated in FIG. 6, the auxiliary medical device 140 is a traction device such as a tether, and the medical device 300 is similar to the medical device 300 illustrated FIG. 5, with an engagement member 310 configured to grasp the auxiliary medical device 140. Similar to the traction device 130 illustrated in FIG. 5, the auxiliary medical device 140 may include a loop 142 to enable the engagement member 310 of the medical device 300 to more readily grasp the auxiliary medical device 140. Various aspects of the interaction of the engagement member 310 with the auxiliary medical device 130 described above are applicable, mutatis mutandis, to the interactions of the engagement member 310 with the auxiliary medical device 140 illustrated in FIG. 6 and FIG. 7 and, for the sake of brevity, are thus not repeated.

Instead of having an end for anchoring to the scaffold system 110, the auxiliary medical device 140 illustrated in FIG. 6 may extend proximally through the auxiliary elongate member 120 for manipulation by a medical professional. The proximal end of the auxiliary medical device 140 may be directly grasped by a medical professional, or coupled to a controller (e.g., control handle or the like) which is grasped by a medical professional to manipulate (e.g., advance or retract) the auxiliary medical device 140 with respect to the auxiliary elongate member 120. The medical device 300 may thus grasp the auxiliary medical device 140 and move the auxiliary medical device 140 to target tissue T to operably couple the auxiliary medical device 140 with the target tissue T, such as with, via, with the assistance of, etc., the engagement member 310 of the medical device 300. Because the scaffold system 110 is movable independently of the elongate member 200, such as discussed above, the scaffold system 110 allows independent movement of the auxiliary elongate member 120 and thus the position of the auxiliary medical device 140 extending therefrom relative to the target tissue T and/or the elongate member 200. In some aspects, the distal end 120d of the auxiliary elongate member 120 may serve as an anchor point for the auxiliary medical device 140, with rotational or axial movement of the auxiliary elongate member 120 affecting the force vector applied by the auxiliary medical device 140 to target tissue T to which the auxiliary medical device 140 is operably coupled.

An additional medical device 400 may be delivered by the elongate member 200 (typically after withdrawal and removal of the first medical device 300) to perform a further aspect or task of a procedure being performed with respect to the target tissue T. In some aspects, a further aspect or task of a procedure is performed with an additional medical device 400 while the auxiliary medical device 140 remains operably coupled with the target tissue T. For instance, as described with reference to the example of an embodiment illustrated in FIG. 5 or FIG. 6, the target tissue T may be cut (e.g., resected, such as in an endoscopic mucosal resection procedure, or dissected, such as in an endoscopic submucosal dissection procedure) by an additional medical device 400 in the form of a cutting device (e.g., blade, knife, scalpel, electrocautery/electrosurgical knife, etc.), such as illustrated in FIG. 7. It will be appreciated that although FIG. 7 illustrates an auxiliary medical device 140 extending from the auxiliary elongate member 120, the descriptions of the auxiliary medical device 140 are applicable to an auxiliary medical device 130 extending from the scaffold system 110 such as illustrated in FIG. 5. Movement of the auxiliary elongate member 120 and the anchor point it defines at its distal end 120d (or the anchor point 131 of the auxiliary medical device 130 of FIG. 5) adjusts the force vector applied by the auxiliary medical device 140 (or the auxiliary medical device 130) to the target tissue T without affecting the position of the elongate member 200 and thus without affecting the position of the additional medical device 400. The auxiliary delivery system 100 thus provides better control and adjustability of traction applied to the target tissue T by the auxiliary medical device 130, 140 and the auxiliary delivery system 100. The magnitude and or direction of traction applied by the auxiliary medical device 130, 140 may be adjusted as needed as the medical device 400 cuts the target tissue T away from the surrounding target site TS. Once the target tissue T is fully removed from the target site TS, the auxiliary medical device 130, 140 may be retracted for better control of the target tissue T coupled to the auxiliary medical device 140 (and removed from the patient). For instance, the target tissue T which has been separated from the target site TS may be held by an instrument, such as a grasper, extended through working channel of the elongate member 200, and retracted along with the instrument holding the tissue to remove the target tissue T from the patient, such as in a manner known to those of ordinary skill in the art.

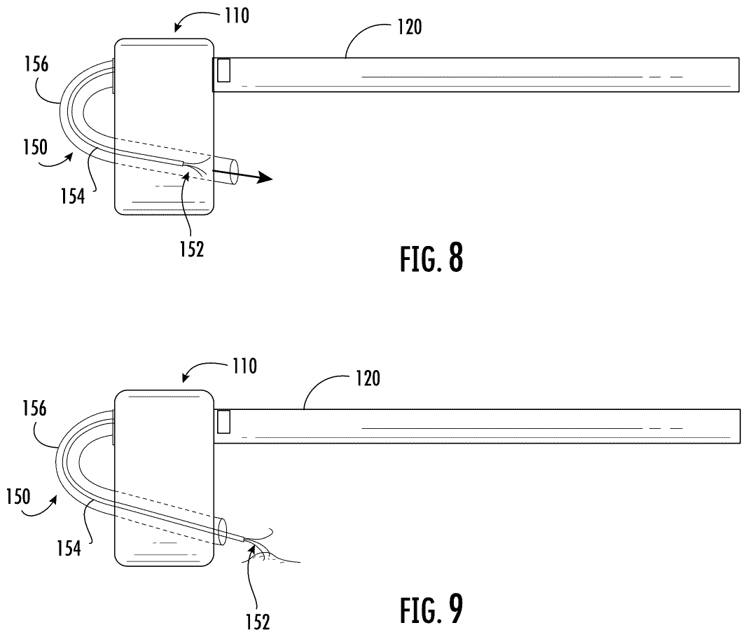

It will be appreciated that an auxiliary delivery system 100 formed in accordance with various principles of the present disclosure may deliver auxiliary medical devices similar to those described above but with various modifications, or other auxiliary medical devices than those described above, such as configured for different procedures. In the example of an embodiment illustrated in FIG. 8 and FIG. 9, the illustrated auxiliary medical device 150 is a traction device capable of engaging tissue without the assistance of an additional device such as an engagement member 310 delivered by the elongate member 200. More particularly, the auxiliary medical device 150 illustrated in FIG. 8 and FIG. 9 has a tissue engagement member 152 formed or provided on an end of a traction element 154 (tether, traction band, suture, string, cord, wire, filament, etc.). The tissue-engagement member 152 may be in the form of an anchor, tines, barbs, etc., or other element capable of engaging with tissue, and remaining engaged with the tissue independently of (e.g., without the assistance of) another device (such as the medical device 300 illustrated in FIG. 5, FIG. 6, or FIG. 7). A sheath 156 may be provided over the tissue-engagement member 152, as illustrated in FIG. 8, to protect tissue from the tissue-engagement member 152 until the tissue-engagement member 152 is at the appropriate location to engage the target tissue T. The tissue-engagement member 152 may then be advanced distally out of the sheath 156, as illustrated in FIG. 9. In some aspects, the tissue-engagement member 152 is made of shape memory material (e.g., nitinol) preformed, such as to have an arch or otherwise bent shape, such as at a distal end thereof. As the preformed bent shape of the tissue-engagement member 152 is pushed distally out of the sheath 156, the tissue-engagement member 152 expands to grab/hold onto tissue. In some aspects, the tissue T can be disengaged by pulling the tissue-engagement member 152 back into the sheath 156. With an auxiliary medical device 150 configured as illustrated in FIG. 8 and FIG. 9, a medical device delivered by the elongate member 200 (such as, without limitation, a medical device 300 such as illustrated in FIG. 5 and FIG. 6), may be used to assist with moving the tissue-engagement member 152 to engage the target tissue T, but need not deploy an additional tissue-engagement member (such as the tissue-engagement member 310 illustrated in FIG. 5, FIG. 6, or FIG. 7) to maintain engagement of the tissue-engagement member 152 with the target tissue T. Any suitable medical device capable of moving the tissue engagement member 152 into engagement with tissue may be used, such as, without limitation, a grasper, forceps, clamp, etc.).

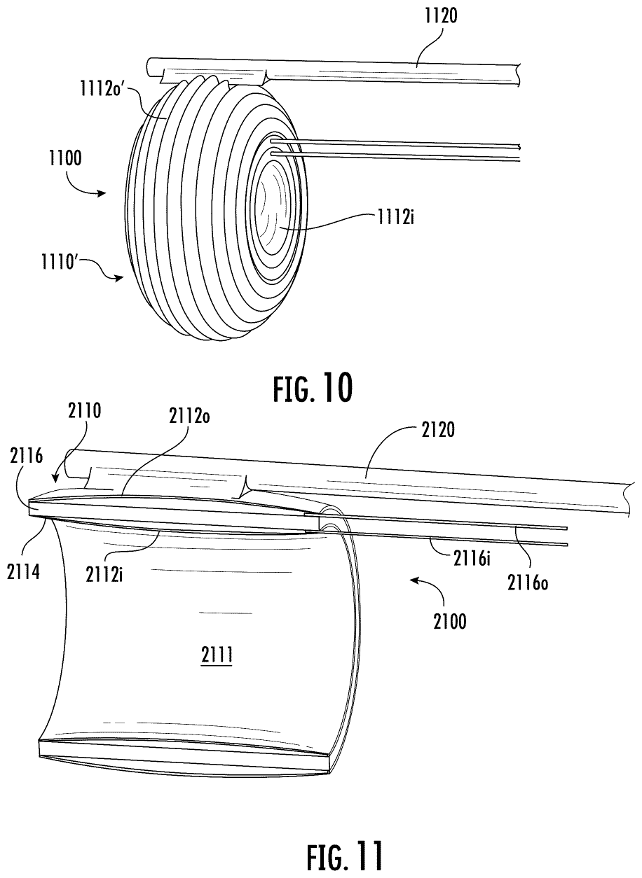

Various modifications may be made to the expandable elements of an auxiliary delivery system formed in accordance with various principles of the present disclosure to enhance the ability of the scaffold system to anchor with respect to the target site TS. For instance, the outer surface of the scaffold system (e.g., the outer surface of the outer expandable element thereof) may be configured to resist, inhibit, prevent, etc. (such terms being used interchangeably herein without intent to limit), migration once the scaffold system (or at least the outer expandable element thereof) is expanded to engage with tissue at the target site TS. In the example of an embodiment of an auxiliary delivery system 1100 illustrated in FIG. 10, the outer surface of the scaffold system 1110′ (e.g., the outer surface of the outer expandable element 1112o′) may be textured, ribbed, flared, grooved, roughened, etc., to prevent migration and/or to enhance engagement with tissue and/or the elongate member 200. In the example of an embodiment illustrated in FIG. 10, the outer surface of the outer expandable element 1112o′ includes ribs. Although the ribs may generally circumferentially extend around the scaffold system 1110′, and may all be in contact with one another and each have rounded outer surfaces, such as illustrated in FIG. 10, it will be appreciated that the geometrical extent (e.g., circumferential, axial, length, width, etc.), number, spacing, relative sizes, contours, etc. of the ribs may be varied in accordance with various principles of the present disclosure, and need not be specifically as illustrated in FIG. 10. It will further be appreciated that texturing other than or in addition to ribs may be provided along a portion or all of the outer surface of the scaffold system.

Various modifications may be made to the structures, elements, mechanisms for expanding/contracting the expandable elements, and/or other features of the scaffold system of an auxiliary delivery system formed in accordance with various principles of the present disclosure. For instance, various modifications to fluid coupling of inflation lumens with expandable elements of a scaffold system formed in accordance with various principles of the present disclosure may be made without departing from the various principles of the present disclosure.

Turning to the example of an embodiment of an auxiliary delivery system 2100 illustrated in FIG. 11, the inflation lumens 2116i, 2116o are fluidly coupled to the expandable elements 2112i, 2112o without being fluidly coupled via the support structure 2114. The support structure 2114 may thus simply provide support to the expandable elements 2112i, 2112o, without fluidly communicating with the inflation lumens 2116i, 2116o for expanding the expandable elements 2112i, 2112o. As such, the support structure 2114 need not be differentiated into an outer support structure and an inner support structure as in the above-described embodiments. The remaining elements and operations of the auxiliary deliver system 2100 may be substantially the same as the above-described embodiments, reference being made to the above descriptions as applying mutatis mutandis to the example of an embodiment illustrated in FIG. 11.

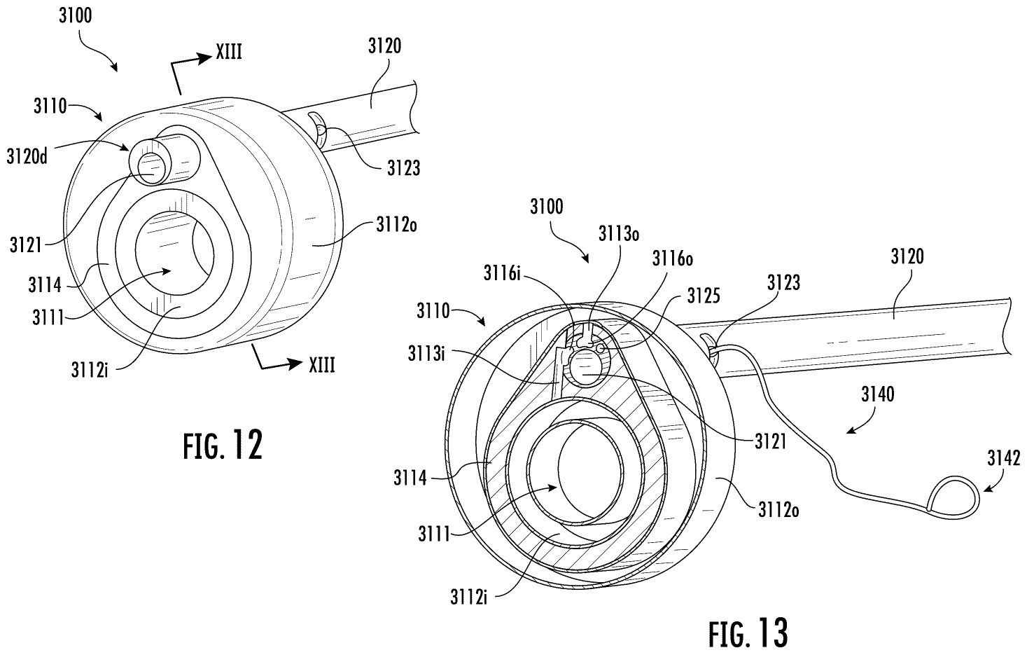

In the example of an embodiment of an auxiliary delivery system 3100 illustrated in FIG. 12 and FIG. 13, instead of a support structure having a separate element for each expandable element of the scaffold system, as in the examples of embodiments illustrated in FIG. 1, FIG. 2, FIG. 2A, FIG. 3, FIG. 3A, FIG. 4, FIG. 4A, FIG. 5, FIG. 6, FIG. 7, FIG. 8, FIG. 9, or FIG. 10, and instead of a support structure which is not fluidly communicated with either of the expandable elements of the scaffold system, a common support structure 3114 for the expandable elements 3112i, 3112o of the illustrated scaffold system 3110 fluidly communicates with both of the expandable elements 3112i, 3112o. More particularly, as may be appreciated with reference to the cross-sectional view illustrated in FIG. 13, along line XIII-XIII of FIG. 12, the support structure 3114 fluidly communicates, via a fluid communication port 3113i, with a lumen 3116i delivering or withdrawing inflation medium with respect to the inner expandable element 3112i, and also communicates, via a fluid communication port 3113o, with a lumen 3116o delivering or withdrawing inflation medium with respect to the outer expandable element 3112o. In the example of an embodiment of an auxiliary delivery system 3100 illustrated in FIG. 12 and FIG. 13, the lumens 3116i, 3116o are defined through the auxiliary elongate member 3120. However, separate elongate members may, instead, define the lumens 3116i, 3116o (such as in the example of an embodiment illustrated in FIG. 1), and the auxiliary elongate member 3120 may be dedicated only to delivery of one or more medical devices.

As illustrated in FIG. 12, the auxiliary elongate member 3120 defines a lumen 3121 therethrough which may open at the distal end 3120d of the auxiliary elongate member 3120. Alternatively or additionally, the lumen 3121 defined in the auxiliary elongate member 3120 may communicate with the exterior of the auxiliary elongate member 3120 radially outwardly through a side opening 3123 through the generally tubular wall defining the auxiliary elongate member 3120. The side opening 3123 may be a skived hole, or simply a radially extending hole, or otherwise, the present disclosure not being limited in this regard. In some aspects, the side opening 3123 is located proximal to the scaffold system 3110 so that an auxiliary medical device 3140 may be delivered proximal to the scaffold system 3110 and thus closer to the elongate member 200 than if delivered out the distal end 3120d of the auxiliary elongate member 3120 after the scaffold system 3110 has been deployed off of the elongate member 200. Optionally, an additional lumen 3125 may be defined through the auxiliary elongate member 3120, such as illustrated in FIG. 13. In some aspects, the additional lumen 3125, instead of the lumen 3121, may communicate with the side opening 3123 for an auxiliary medical device 3140 to be delivered proximal to the scaffold system 3110. In such embodiment, the lumen 3121 may deliver a different auxiliary medical device out the distal end of the auxiliary elongate member 3120. An optional loop 3142 may be provided at the distal end of the auxiliary medical device 3140 and grasped by an engagement member of a medical device (such as the engagement member 310 delivered by the elongate member 200 illustrated in FIG. 5) without the need to extend the medical device through the lumen 3111 of the scaffold system 3110.

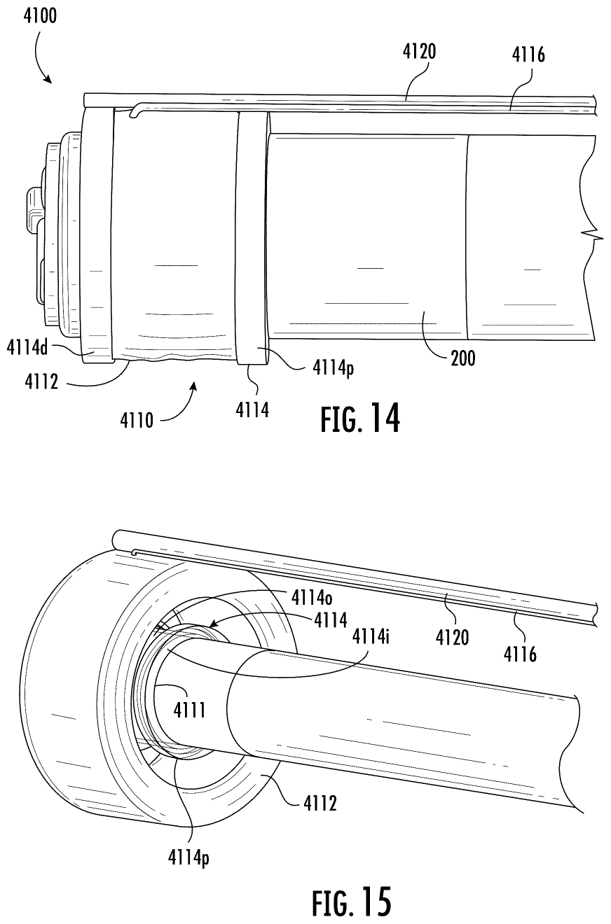

In some aspects, instead of an inner expandable element and an outer expandable element, a scaffold system formed in accordance with various principles of the present disclosure may have only one expandable element. For instance, in the example of an embodiment of an auxiliary delivery device and system 4100 illustrated in FIG. 14 and FIG. 15, the scaffold system 4110 includes an expandable element 4112 positioned over (e.g., on, around, circumferentially around, etc.) a support structure 4114. It is noted that the proportions of the auxiliary delivery device and system 4100 illustrated in FIG. 15 are smaller than in FIG. 14. The example of an embodiment of a support structure 4114 is mounted over the exterior of the elongate member 200, and the expandable element 4112 is delivered over the support structure 4114 and the elongate member 200 to a target site. In some aspects, the support structure 4114 is substantially fixedly coupled to the elongate member 200 (e.g., fixed against at least axial translation therewith, and optionally against rotational movement as well). In some aspects, the support structure 4114 is formed with a seat 4114o along an outer surface thereof. The expandable element 4112 may be seated in the seat 4114o during delivery of the scaffold system 4110 to a target site. Typically, the expandable element 4112 is delivered in a generally unexpanded configuration to facilitate delivery within the body without adding significantly to the outer dimensions of the elongate member 200 and support structure 4114. In some aspects, the support structure 4114 is generally cylindrical with a lumen defining the lumen 4111 of the scaffold system 4110 through which the elongate member 200 may extend. In some aspects, the support structure 4114 may include a proximal shoulder 4114p and a distal shoulder 4114d at each axial end of the seat 4114o to retain the outer expandable element 4112o axially in place with respect to the support structure 4114 to prevent, inhibit, limit, etc., relative axial shifting of the expandable element 4112 with respect to the support structure 4114 and/or the elongate member 200.

The expandable element 4112o of the example of an embodiment of a scaffold system 4110 illustrated in FIG. 14 and FIG. 15 may be expanded, as illustrated in FIG. 15, to increase in diameter (both inner and outer diameter), such as to expand away from the scat 4114o. The expandable element 4112 may thereby be moved off of the elongate member 200 to be moved independently of the elongate member 200. As may be appreciated, the expandable element 4112 may be expanded by delivery of an inflation medium via an inflation lumen 4116 similar to the previously described inflation lumens 116, 2116, 3116. It will be appreciated that instead of the inflation lumen 4116 fluidly communicating with the expandable element 4112 along an outer surface of the expandable element 4112 (as illustrated in FIG. 14 and FIG. 15), the inflation lumen 4116 may fluidly communicate with the expandable element 4112 along a proximal end thereof (such as, for example, the inflation lumen 2116o communicating with a proximal end of the outer expandable element 2112o as in any of the above-described examples of embodiments).

In some aspects, the expandable element 4112 is movable axially relative to the elongate member 200, such as to be positioned distal to the elongate member 200. The expandable element 4112 may be inflated sufficiently to engage with tissue walls (e.g., inner tissue walls of a body lumen) to anchor the expandable element 4112 with respect to a target site within a patient's body. In some aspects, the expandable element 4112 is movable rotationally with respect to the elongate member 200, such as to adjust the angular position of the auxiliary elongate member 4120 relative to the elongate member 200. In some aspects, the expandable element 4112 may be inflated to anchor with respect to tissue, and then deflated to facilitate rotation of the expandable element 4112 relative to the tissue and the elongate member 200. As described above with reference to the other above-described examples of embodiments, the auxiliary elongate member 4120 may be configured to deliver a medical device in addition to a medical device delivered by the elongate member 200. For the sake of brevity, and without intent to limit, reference is made to the above descriptions of delivery of medical devices as optionally applying mutatis mutandis to the example of an embodiment of an auxiliary delivery system 4100 illustrated FIG. 14 and FIG. 15.

The examples of embodiments of scaffold systems formed in accordance with various principles of the present disclosure which have been described thus far have expandable elements which are expanded with an inflation medium, and selectively deflated as desired. However, in some aspects, the expandable elements of a scaffold system formed in accordance with various principles of the present disclosure are not expanded with an inflation medium. For instance, the expandable elements of a scaffold system formed in accordance with various principles of the present disclosure may shift in configuration, shape, position, etc., between an unexpanded configuration and an expanded configuration. In the unexpanded configuration, the expandable elements may occupy minimal space radially. In the expanded configuration, the expandable elements may shift radially, such as by bowing, flexing, etc., such as to increase a radial dimension of the scaffold system.

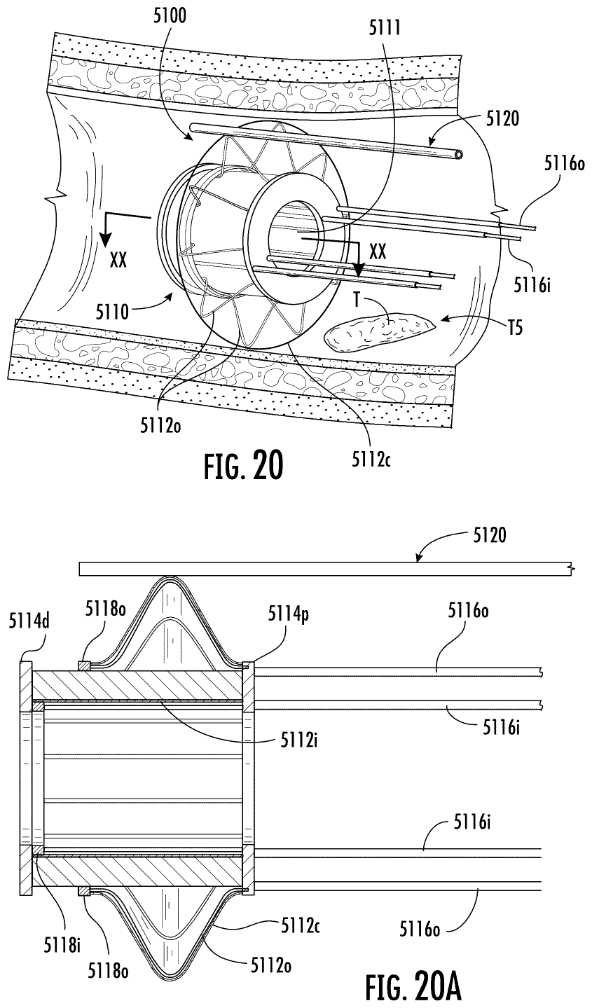

An example of an embodiment of an auxiliary delivery system 5100 with a scaffold system 5110 having expandable elements 5112i, 5112o shifting between expanded and unexpanded configurations without the use of an inflation medium is illustrated in FIG. 16. It will be appreciated that the scaffold system 5110 is illustrated distal to an elongate member 200 over which the scaffold system 5110 may be delivered to a target site within a patient's body to facilitate illustration of the inner expandable element 5112i. In some aspects, the inner expandable element 5112i includes one or more inner expandable elements 5112i. For the sake of convenience, and without intent to limit, reference is made to the inner expandable element 5112i in the singular form, even though two or more inner expandable elements 5112i may be provided. Alternatively or additionally, in some aspects, the outer expandable element 5112o includes one or more spaced apart elongated outer expandable elements 5112o. For the sake of convenience, and without intent to limit, reference is made to the outer expandable element 5112o in the singular form, even though two or more spaced apart elongated outer expandable elements 5112o may be provided. In some aspects, a silicone cover is provided over all or some of the spaced apart elements of one or both of the expandable elements 5112i, 5112o. An example of an embodiment of a silicone cover 5112c is illustrated in FIG. 20 over the plurality of spaced apart elements of the outer expandable element 5112o. A similar silicone cover may be provided over the plurality of spaced apart elements of the inner expandable element 5112i but, to simplify the illustrations of the inner expandable element 5112i, is not illustrated.

As illustrated in FIG. 16, showing a distal view of the scaffold system 5110, and in

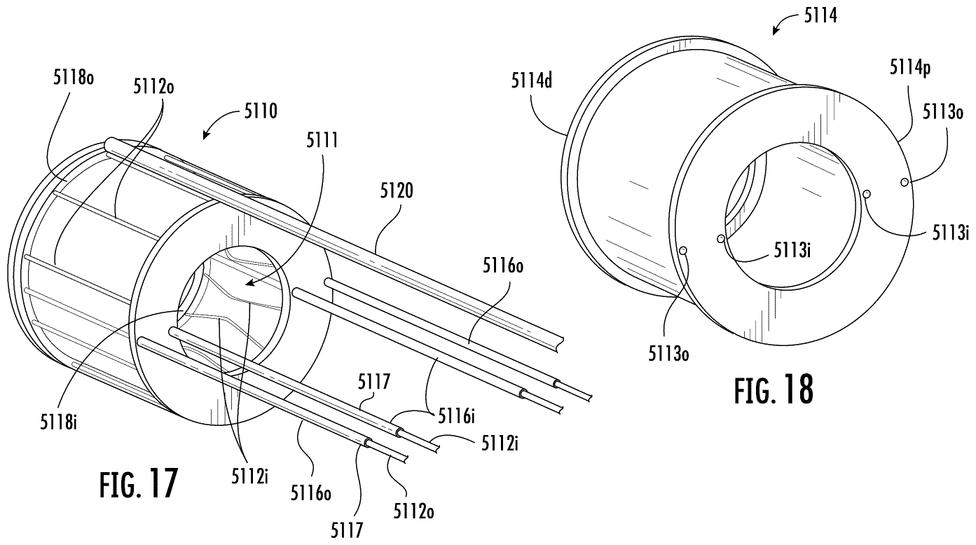

FIG. 17 showing a proximal view of the scaffold system 5110, the inner expandable element 5112i includes one or more inner expandable elements 5112i which are bowed or flexed radially inwardly into the lumen 5111 of the scaffold system 5110. In such configuration, which may be considered a delivery configuration of the inner expandable element 5112i, the inner expandable element 5112i effectively reduces the inner diameter of the lumen 5111 of the scaffold system 5110 to engage the outer surface of an elongate member 200 extended within the lumen 5111. In the delivery configuration of the outer expandable element 5112o illustrated in FIG. 16 and FIG. 17, the outer expandable element 5112o extends generally longitudinally, with little to no bowing. In some aspects, in the delivery configuration of the scaffold system 5110, the outer expandable element 5112o does not extend beyond the outer dimensions of the support structure 5114 so that the outer dimensions of the outer expandable element 5112o do not add to the overall outer dimensions of the auxiliary delivery system 5100 and the outer expandable element 5112o does not interfere with movement of the elongate member 200 within the patient's body.

In accordance with various principles of the present disclosure, the inner expandable element 5112i and the outer expandable element 5112o of the scaffold system 5110 illustrated in FIG. 16 and FIG. 17 are shiftable between expanded and unexpanded configurations independently of each other. To separate the scaffold system 5110 from the elongate member 200 (such as upon reaching a target site within a patient), the inner expandable element 5112i may be shifted to a generally elongated deployed configuration straighter than the configuration illustrated in FIG. 16 and FIG. 17 (such as illustrated in FIG. 20, discussed in further detail below) to increase the diameter of the lumen 5111 of the scaffold system 5110 to greater than the outer diameter of the elongate member 200. The outer expandable element 5112o may remain in a configuration such as illustrated in FIG. 16 and FIG. 17 once the scaffold system 5110 is separated from the elongate member 200 (with the inner expandable element 5112i in the deployed configuration), or may be shifted to a radially outwardly bowed or flexed configuration (such as illustrated in FIG. 20, and discussed in further detail below), such as a deployed configuration engaging tissue at a target site within a patient.

In some aspects, the inner expandable element 5112i and/or the outer expandable element 5112o is actuatable (between a delivery configuration and a deployed configuration) by one or more actuator elements 5116 which extend proximally from the scaffold system 5110 for control by a medical professional (either by direct engagement or via a controller such as a handle coupled to the actuator element 5116). In some aspects, if one or both of the expandable elements 5112i, 5112o each include two or more expandable elements 5112i, 5112o, an actuator element 5116 may be associated with all or just some but not all of the elements of expandable elements 5112i, 5112o. The support structure 5114 along which the expandable elements 5112i, 5112o extend is illustrated in isolation in FIG. 18 to provide a clearer illustration of the apertures 5113i, 5113o through which the actuator elements 5116 are operably associated with the expandable elements 5112i, 5112o. One or more inner actuator elements 5116i are each operably associated with a respective inner expandable element 5112i to shift the inner expandable element 5112i between a generally elongated configuration and a bowed configuration. Similarly, one or more outer actuator elements 5116o are each operably associated with a respective outer expandable elements 5112o to shift the outer expandable element 5112o between a generally elongated configuration and a bowed configuration. In some aspects, the one or more actuator elements 5116 are coupled with a support ring 5118i, 5118o which shifts the configurations of expandable elements 5112i, 5112o coupled thereto (as described in further detail below). In some aspects, one or more of the actuator elements 5116 may be in the form of a sheath 5117 defining a lumen through which one of the expandable elements 5112i, 5112o slidably extends. In some aspects, the expandable element 5112i, 5112o extending through the sheath 5117 is axially translatable within and with respect to the sheath 5117 to actuate the portion of such expandable element 5112i, 5112o extending along the support structure 5114. Movement of the expandable elements 5112i, 5112o extending through a respective sheath 5117 may be controlled by a medical professional manipulating (e.g., axially translating) the proximal end thereof (such as by directly grasping the proximal end or by manipulating a control handle, or the like, which is coupled with the expandable elements 5112i, 5112o).

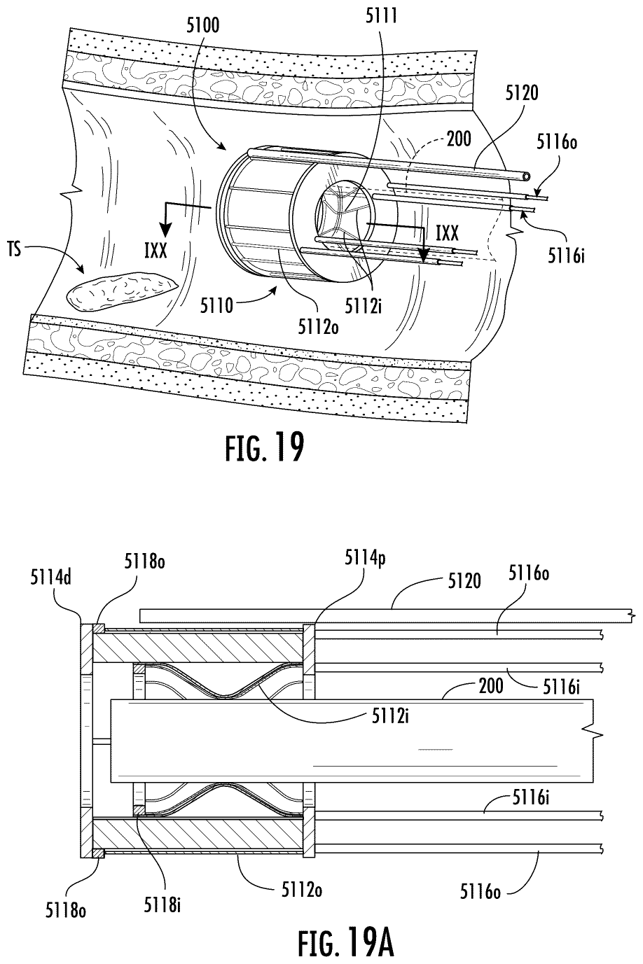

In the example of an embodiment of a scaffold system 5110 illustrated in FIG. 16, FIG. 17, FIG. 18, FIG. 19, FIG. 19A, FIG. 20, and FIG. 20A, the proximal end of one or more of the expandable elements 5112i, 5112o is coupled to a proximal shoulder 5114p of the support structure 5114. The illustrated proximal shoulder 5114p includes a radially-inwardly extending shoulder and a radially-outwardly extending shoulder, each extending transverse to the longitudinal axis of the support structure 5114. The distal ends of expandable elements 5112i, 5112o are coupled to a respective support ring 5118i, 5118o which may extend circumferentially with respect to the support structure 5114. In an unexpanded configuration, the support ring 5118i, 5118o is positioned adjacent a distal shoulder 5114d of the support structure 5114. Proximal movement (e.g., pulling) of one or more of the actuator elements 5116 (such as an actuator element 5116 coupled to a support ring 5118i, 5118o and/or an expandable elements 5112i, 5112o extending proximally through a sheath 5117 of an actuator element 5116) pulls the support ring 5118i, 5118o coupled thereto proximally towards a proximal shoulder 5114p of the support structure 5114. As a support ring 5118i, 5118o moves proximally, the expandable element 5112i, 5112o operably associated therewith bows away from the support structure 5114 (radially inwardly for the inner expandable elements 5112i and radially outwardly for the outer expandable elements 5112o).

The operation of the auxiliary delivery device and system 5100 may be better appreciated with reference to FIG. 19, FIG. 19A, FIG. 20, and FIG. 20A. As illustrated in FIG. 19, the inner expandable element 5112i is bowed in a delivery configuration to engage the exterior of the elongate member 200. In the illustrated delivery configuration, the inner expandable element 5112i substantially fixes the scaffold system 5110 with respect to the elongate member 200 to extend therewith within a patient's body for delivery to a target site TS. Furthermore, the outer expandable element 5112o remains generally elongated (e.g., in a delivery configuration thereof) so as not to interfere with delivery of the auxiliary delivery system 5100. As may be more readily appreciated with reference to the cross-sectional view along line XIX-XIX of FIG. 19 illustrated in FIG. 19A, the inner support ring 5118i is proximally spaced from the distal shoulder 5114d of the support structure 5114. In contrast, the outer support ring 5118o is closer to the distal shoulder 5114d than the inner support ring 5118i is to the distal shoulder 5114d. Thus, the outer expandable element 5112o is more elongated and straighter than the inner expandable element 5112i, and the inner expandable element 5112i is more bowed than the outer expandable element 5112o.

Once the auxiliary delivery device and system 5100 have reached the target site TS, the inner actuator element 5116i may be actuated to shift the inner expandable element 5112i to a deployed configuration disengaging the exterior of the elongate member 200 to allow the scaffold system 5110 to be released from the elongate member 200. The auxiliary delivery device and system 5100 is illustrated in FIG. 20 as advanced distally off of the elongate member (shown in FIG. 19). As may be appreciated with reference to the cross-sectional view of FIG. 20 (along line XX-XX) illustrated in FIG. 20A, the inner support ring 5118i is positioned distal to the position in which the inner support ring 5118i is positioned in FIG. 19A, and closer to the distal shoulder 5114d of the support structure 5114 so that the inner expandable element 5112i is straighter/less bowed than in FIG. 19A. In the configuration illustrated in FIG. 20 and FIG. 20A, the inner expandable element 5112i increases the space (e.g., cross-sectional area and/or diameter) within the lumen 5111 of the scaffold system 5110 to allow the scaffold system 5110 to be distally advanced off of the elongate member 200 to be deployed. As may also be appreciated with reference to FIG. 20 and FIG. 20A, the outer support ring 5118o is proximally spaced from the distal shoulder 5114d of the support structure 5114. Thus, the outer expandable element 5112o is bowed radially outwardly from the support structure 5114. The outer expandable element 5112o may be sufficiently bowed to engage tissue at the target site TS, such as the inner wall of a body lumen in which the auxiliary delivery system 5100 is positioned. In such configuration, the scaffold system 5110 may be considered to be in a deployed and engaged and/or anchored configuration with respect to the target tissue T, with the outer expandable element 5112o in a deployed configuration anchoring the auxiliary delivery device 5100 with respect to the target site TS and the target tissue T. An auxiliary medical device such as shown in any of the previously described embodiments may be delivered with the auxiliary delivery device and system 5100, in any of the above-described manners. Moreover, as described above with reference to any of the above-described embodiments, the position of the auxiliary delivery device and system 5100 may be adjusted relative to the elongate member 200 without affecting the position of the elongate member 200. For instance, the outer expandable element 5112o may be moved to a straighter configuration, such as by moving the outer support ring 5118o distally toward the distal shoulder 5114d of the support structure 5114. Once the outer expandable element 5112o is moved a sufficient distance from the tissue wall (with respect to which the scaffold system 5110 was anchored) to freely move with respect to the tissue wall, the scaffold system 5110 may be axially advanced or retracted, or rotated, or otherwise moved with respect to the elongate member 200.

It will be appreciated that although the above disclosure generally describes an auxiliary delivery system delivered distal to a target site, the auxiliary delivery system may, instead, be delivered proximal to a target site, with accompanying modifications to use thereof as may be appreciated by those of ordinary skill in the art. Moreover, it will be appreciated that although the above disclosure generally describes an auxiliary delivery system delivered distal to an elongate member such as an endoscope with a procedure performed with the endoscope remaining proximal to the auxiliary delivery system, the elongate member may be advanced (e.g., through the auxiliary delivery system, or at least the scaffold system thereof) to be distal to the auxiliary delivery system, such that a procedure is performed with the endoscope distal to the auxiliary delivery system. As may be appreciated, if an auxiliary delivery system is to apply traction to tissue, the traction may be applied in a distal or proximal direction, depending on the relative positions of the auxiliary delivery system, the target site, and/or the elongate member. Similarly, as may be appreciated, if an auxiliary delivery system delivers an auxiliary medical device, the auxiliary medical device may be operated distal or proximal to the target site and/or the elongate member, depending on the relative positions of the auxiliary delivery system, the target site, and/or the elongate member.

In accordance with various principles of the present disclosure, devices and systems formed in accordance with various principles of the present disclosure, and associated methods, preferably do not functionally or operationally impact (e.g., significantly increase) the outer profile, such as the outer diameter, of an elongate member over which the devices, systems, and methods are provided. For instance, navigation of an elongate member within the body is not negatively impacted by addition of a device or system formed in accordance with various principles of the present disclosure. Moreover, devices and systems formed in accordance with various principles of the present disclosure add minimal additional bulk to the pre-existing elongate member during navigation to a treatment site.

It will be appreciated that different sizes, configurations, arrangements, etc., of components of an auxiliary delivery device and system formed in accordance with various principles of the present disclosure may be used depending on the procedure, the nature of the target site (e.g., location, area/size, type of tissue, etc.), preferences of the medical professional, and other considerations relevant to a given procedure.

In view of the above, it should be understood that the various embodiments illustrated in the figures have several separate and independent features, which each, at least alone, has unique benefits which are desirable for, yet not critical to, the presently disclosed devices and systems. For example, various features of the disclosure are grouped together in one or more aspects, embodiments, or configurations for the purpose of streamlining the disclosure. Therefore, the various separate features described herein need not all be present in order to achieve at least some of the desired characteristics and/or benefits described herein. Only one of the various features may be present in a device or system formed in accordance with various principles of the present disclosure. Alternatively, one or more of the features described with reference to one embodiment can be combined with one or more of the features of any of the other embodiments provided herein. That is, any of the features described herein can be mixed and matched to create hybrid designs, and such hybrid designs are within the scope of the present disclosure. Moreover, various features hereinafter described may be used singly or in any combination thereof.

Various further benefits of the various aspects, features, components, and structures of a delivery system and associated devices and methods such as described above, in addition to those discussed above, may be appreciated by those of ordinary skill in the art. It should be understood that the foregoing discussion has broad application and has been presented for purposes of illustration and description and is not intended to limit the disclosure to the form or forms disclosed herein. It is to be understood by one of ordinary skill in the art that the present discussion is a description of illustrative examples of embodiments only, and is not intended as limiting the broader aspects of the present disclosure. Thus, references to elements or structures or features in the drawings must be appreciated as references to examples of embodiments of the disclosure, and should not be understood as limiting the disclosure to the specific elements, structures, or features illustrated. Various additions, modifications, and substitutions may be made to embodiments disclosed herein without departing from the concept, spirit, and scope of the present disclosure. In particular, it will be clear to those skilled in the art that principles of the present disclosure may be embodied in other forms, structures, arrangements, proportions, and with other elements, materials, and components, without departing from the concept, spirit, or scope, or characteristics thereof. One skilled in the art will appreciate that the disclosure may be used with many modifications or modifications of structure, arrangement, proportions, materials, components, and otherwise, used in the practice of the disclosure, which are particularly adapted to specific environments and operative requirements without departing from the principles or spirit or scope of the present disclosure. For example, elements shown as integrally formed may be constructed of multiple parts or elements shown as multiple parts may be integrally formed, the operation of elements may be reversed or otherwise varied, the size or dimensions of the elements may be varied. Similarly, while operations or actions or procedures are described in a particular order, this should not be understood as requiring such particular order, or that all operations or actions or procedures are to be performed, to achieve desirable results. Additionally, other implementations are within the scope of the following claims. In some cases, the actions recited in the claims can be performed in a different order and still achieve desirable results. The presently disclosed embodiments are therefore to be considered in all respects as illustrative and not restrictive, the scope of the claimed subject matter being indicated by the appended claims, and not limited to the foregoing description or particular embodiments or arrangements described or illustrated herein. In view of the foregoing, individual features of any embodiment may be used and can be claimed separately or in combination with features of that embodiment or any other embodiment, the scope of the subject matter being indicated by the appended claims, and not limited to the foregoing description.

In the foregoing description and the following claims, the following will be appreciated. The phrases “at least one”, “one or more”, and “and/or”, as used herein, are open-ended expressions that are both conjunctive and disjunctive in operation. The terms “a”, “an”, “the”, “first”, “second”, etc., do not preclude a plurality. For example, the term “a” or “an” entity, as used herein, refers to one or more of that entity. As such, the terms “a” (or “an”), “one or more” and “at least one” can be used interchangeably herein. As used in this specification and the appended claims, the term “or” is generally employed in its sense including “and/or” unless the content clearly dictates otherwise. As used herein, the conjunction “and” includes each of the structures, components, features, or the like, which are so conjoined, unless the context clearly indicates otherwise, and the conjunction “or” includes one or the others of the structures, components, features, or the like, which are so conjoined, singly and in any combination and number, unless the context clearly indicates otherwise. All directional references (e.g., proximal, distal, upper, lower, upward, downward, left, right, lateral, longitudinal, front, back, top, bottom, above, below, vertical, horizontal, radial, axial, clockwise, counterclockwise, and/or the like) are only used for identification purposes to aid the reader's understanding of the present disclosure, and/or serve to distinguish regions of the associated elements from one another, and do not limit the associated element, particularly as to the position, orientation, or use of this disclosure. Connection references (e.g., attached, coupled, connected, engaged, joined, etc.) are to be construed broadly and may include intermediate members between a collection of elements and relative movement between elements unless otherwise indicated. As such, connection references do not necessarily infer that two elements are directly connected and in fixed relation to each other. Identification references (e.g., primary, secondary, first, second, third, fourth, etc.) are not intended to connote importance or priority, but are used to distinguish one feature from another.

The following claims are hereby incorporated into this Detailed Description by this reference, with each claim standing on its own as a separate embodiment of the present disclosure. In the claims, the terms “comprises”, “comprising”, “includes”, and “including” do not exclude the presence of other elements, components, features, groups, regions, integers, steps, operations, etc. Additionally, although individual features may be included in different claims, these may possibly advantageously be combined, and the inclusion in different claims does not imply that a combination of features is not feasible and/or advantageous. In addition, singular references do not exclude a plurality. Reference signs in the claims are provided merely as a clarifying example and shall not be construed as limiting the scope of the claims in any way.

What is claimed is:

1. A delivery system for delivering a medical device within a patient's body, said system comprising:
a scaffold system configured to be mounted with respect to an elongate member for delivery therewith to a target site within the patient's body, and separable from the elongate member for axial and rotational movement independent of the elongate member; and
at least one actuator element operably associated with said scaffold system to shift at least one element of said scaffold system between a delivery configuration in which said scaffold system is engageable with the elongate member to move with the elongate member, and a deployed configuration in which said scaffold system and the elongate member are capable of moving axially and rotationally independently of each other.

2. The delivery system of claim 1, wherein said scaffold system comprises at least one expandable element configured to shift between a delivery configuration engageable with the elongate member to move with the elongate member, and a deployed configuration in which said expandable element and the elongate member are capable of moving axially and rotationally independently of each other.

3. The delivery system of claim 2, wherein said at least one expandable element comprises an inner expandable element and an outer expandable element, wherein said inner expandable element and said outer expandable element are independently actuatable between a delivery configuration and a deployed configuration.

4. The delivery system of claim 3, wherein said scaffold system further comprises a support structure defining a lumen, said inner expandable element positioned along an interior of said support structure and said outer expandable element positioned within the lumen of said support structure and defining a lumen configured to receive an elongate member therethrough.

5. The delivery system of claim 2, wherein:
the delivery configuration of said at least one expandable element is a radially inwardly expanded configuration reducing an inner diameter of the lumen of said at least one expandable element to be engageable with the exterior of the elongate member extended therethrough to move with the elongate member, and
the deployed configuration of said at least one expandable element is substantially elongated to increase the inner diameter of the lumen of said at least one expandable element to separate from the elongate member extended therethrough when said at least one expandable element is in the delivery configuration.

6. The delivery system of claim 5, wherein said at least one expandable element is inflatable into the delivery configuration.

7. The delivery system of claim 5, wherein said at least one expandable element is bowed radially inwardly in the delivery configuration and substantially elongated in the deployed configuration.

8. The delivery system of claim 5, further comprising a support structure configured to be mounted on the elongate member to move therewith, said support structure defining a seat for said at least one expandable element, said at least one expandable element configured to engage and be seated in said seat of said support structure to move with said support structure and the elongate member when said at least one expandable element is in the delivery configuration, and is expanded away from said seat of said support structure to disengage said seat of said support structure to be axially and radially movable with respect to said support structure and the elongate member.

9. The delivery system of claim 1, further comprising an elongate member having a distal end coupled to said scaffold system and a proximal end extending proximal for access by a medical professional.

10. The delivery system of claim 9, wherein said elongate member is tubular and configured to deliver an auxiliary medical device with said scaffold system, wherein said tubular elongate member is movable with said scaffold system to move the auxiliary medical device axially or rotationally with respect to an elongate member with which said delivery system is delivered.

11. A delivery system configured to deliver an auxiliary medical device within a patient's body, said system comprising:
a delivery device configured to be delivered with an elongate member configured to deliver a medical device within a patient's body, and to deliver an auxiliary medical device which may interact with the medical device delivered by the elongate member;
wherein said delivery device is movable between a delivery configuration in which said delivery device is mounted with respect to an elongate member to move therewith, and a deployed configuration in which said delivery device and the elongate member are axially and rotatably movable with respect to each other.

12. The delivery system of claim 11, wherein said delivery device comprises a scaffold system configured to be mounted with respect to an elongate member for delivery therewith to a target site within the patient's body, and separable from the elongate member for movement independent of the elongate member.

13. The delivery system of claim 11, wherein said auxiliary medical device is a medical device with a first end anchored to said scaffold system and a second end configured to be engaged with tissue within a patient's body.

14. The delivery system of claim 11, wherein said delivery device comprises a tubular elongate member configured to deliver the auxiliary medical device.

15. The delivery system of claim 14, wherein said tubular elongate member is movable to move said scaffold system axially or rotationally.

16. The delivery system of claim 11, wherein said auxiliary medical device is a traction device, and movement of said delivery device adjusts the force vector said traction device may apply when engaged with tissue.

17. A method of delivering a scaffold system to a target site within a patient's body, said method comprising:
mounting the scaffold system on an elongate member to be movable with the elongate member;
delivering the scaffold system with a distal end of the elongate member to the target site;
shifting the configuration of the scaffold system to be released from the elongate member; and
moving at least one of the scaffold system or the elongate member axially and rotationally with respect to the other of the scaffold system or the elongate member.

18. The method of claim 17, further comprising anchoring the scaffold system with respect to tissue at the target site.

19. The method of claim 17, further comprising expanding a first expandable element of the scaffold system to engage the elongate member to mount the scaffold system to the elongate member, and shifting the expandable element to an unexpanded configuration to release the scaffold system from the elongate member.

20. The method of claim 19, further comprising expanding the first expandable element of the scaffold system or a second expandable element of the scaffold system to anchor the scaffold system with respect to tissue at the target site.
